(19) United States
(12) Patent Application Publication
(10) Pub. No.: US 2003/0027332 A1
(43) Pub. Date: Feb. 6, 2003

(54) Tissue engineered heart valve

(75) Inventors: Hugues Lafrance, Mission Viejo, CA (US); Francois Bergeron, Sainte-Foy (CA); Charles Roberge, Sillery (CA); Lucie Germain, St-Augustin-les-Demaures (CA); Francois Auger, Sillery (CA)

Correspondence Address: John Christopher James, Edward Lifesciences LLC, One Edwards Way, Irvine, CA, 90266, US

(73) Assignee: Edwards Lifesciences Corporation, Irvine, CA

(21) Appl. No.: 10/198,628

(22) Filed: Jul. 16, 2002

Related U.S. Application Data
(60) Provisional application No. 60/306,058, filed on Jul. 16, 2001.

(51) Int. Cl. A61K 48/00; C12N 5/08

(52) U.S. Cl. 435/366; 424/93.21

(57) ABSTRACT

The tissue-engineered heart valve of the present invention is comprised of elements, such as leaflets, formed from self-supporting human engineered tissue. Such self-supporting tissue is comprised of living biological cells and extracellular matrix without the presence of nonviable scaffolding structures. Thus, the tissue-engineered heart valve of the present invention consists of totally living human tissue which could theoretically function like a native biological structure with the potential to grow, to repair and to remodel.

### CROSS-REFERENCES TO RELATED APPLICATIONS

This application claims priority to U.S. Provisional Application No. 60/306058 (Attorney Docket No. 20553D-004300US), filed on Jul. 16, 2001, the full disclosure of which is incorporated herein by reference. This application is also related to Application No. ______ , entitled “Method for Making Multi-Layered Engineered Tissue” (Attorney Docket No. 86194-22), filed on the same day, Jul. 16, 2002, as the instant application and incorporated by reference for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### FIELD OF THE INVENTION

The present invention relates to engineered human tissue. In particular, the present invention relates to cardiac tissue replacement. The present invention also relates to cardiac valve repair and replacement. More particularly, the present invention relates to tissue-engineered living human cardiac valves and methods of making such valves.

### BACKGROUND

Over 150,000 surgical procedures are performed each year to replace damaged or diseased cardiac valves worldwide. In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary valves. Prosthetic heart valves can be used to replace any of these naturally occurring valves. The majority of the replacement procedures currently employ mechanical valve prostheses. Mechanical valves include caged-ball valves (such as Starr-Edwards valves), bi-leaflet valves (such as St. Jude valves) and tilting disk valves (such as Medtronic-Hall or Omniscience valves). Operating much like a rigid mechanical check valve, mechanical heart valves are robust and long lived. Thus, the main advantage of mechanical valves is their long-term durability. However, currently available mechanical valves suffer from the disadvantage that they are thrombogenic and thus the patient requires lifetime anticoagulant therapy. If blood clots form on the valve, they can preclude the valve from opening or closing correctly, or more importantly, the blood clots can disengage from the valve and embolize to the brain causing a stroke, or occlude coronaries causing permanent damages to the myocardium. Anticoagulant drugs can be administered to reduce the risk of blood clot formation, however such drugs are expensive and potentially dangerous in that they can cause abnormal bleeding which, in itself, can cause a stroke if the bleeding occurs within the brain. In addition, they also generate a clicking noise when the mechanical closure seats against the associated valve structure at each beat of the heart.

One alternative to mechanical valves is tissue-type or “bioprosthetic” valves. Bioprosthetic valves are generally made from naturally-derived xenogeneic tissues fixed with glutaraldehyde-based processes. Currently available bioprosthetic valves are constructed either by sewing pig aortic valves to a stent to hold the leaflets in proper position, or by constructing valve leaflets using pericardial sac, such as bovine-derived pericardium, and sewing the leaflets to a stent. The stents can be rigid or slightly flexible and are covered with cloth, usually a synthetic material sold under the trademark Dacron™. The stent is usually attached to a sewing ring for fixation to the patient's native tissue. Such bioprostheses imitate the action of flexible natural heart valve leaflets, which coapt between adjacent tissue junctions known as free edges. Thus, artificial valves constructed from natural tissues have superior hemodynamic characteristics. In addition, tissue-type valve leaflets are flexible, silent, and do not require the use of systemic anticoagulation because they do not cause blood clots to form as readily as do the mechanical valves. However, the major disadvantage of bioprosthetic valves is that they lack the long-term durability of mechanical valves. Naturally occurring processes within the human body can attack and stiffen or “calcify” the tissue leaflets of the valve over time, particularly at high-stress areas of the valve such as at the commissure junctions between the valve leaflets and at the peripheral leaflet attachment points or “cusps” at the outer edge of each leaflet. Further, the valves are subject to stresses from constant mechanical operation within the body. Accordingly, the valves wear out over time and need to be replaced. Thus, currently available tissue valves have a shorter lifetime than mechanical valves, usually requiring replacement at approximately 21 years for porcine-derived valves and 17 years for bovine pericardial valves. As a result, bioprosthetic valves are usually not recommended for patients under the age of 65, or if recommended would likely require to consider another open-heart surgery.

Thus, there is a need for a tissue valve replacement that has long-term durability and is biocompatible with the host, particularly for patients below 65 years of age. To achieve these goals, tissue engineering methods have developed. Tissue engineering in heart valve therapy is a new approach to fabricate a functional heart valve from human cells. Historically, tissue-engineering approaches have relied on decellularized tissues, synthetic or bioresorbable man-made polymers to provide a scaffolding effect and mechanical strength. To create a valve, one basic idea may involve autologous cell seeding of a biocompatible and biodegradable scaffold that is shaped like a heart valve. Once the cells have become attached to the scaffold, they form their own extracellular matrix while the polymer scaffold starts to degrade. With this approach, it is theoretically possible to generate an autologous tissue-engineered heart valve that can be implanted into the same patient from whom the cells were harvested. Such an implantable tissue-engineered structure would have certain potential advantages over the current heart valve substitutes, such as glutaraldehyde-fixed xenografts, mechanical valves and homografts.

The major disadvantage of current tissue-engineered valves is that they consist of foreign body material and are nonviable. Typical scaffolding materials are either polymers composed of chemical substances like poly-glycolic acid, poly-4-hydroxybutyrate, polyhydroxyalkanoate and gels out of extracellular matrix proteins such as collagen or fibrin. Unfortunately, these materials are still far from ideal. They are expensive, potentially immunogenic and further they show toxic degradation and inflammatory reactions. In addition, they might be of poor resorbability. As a consequence, there is a lack of growth and risk of thromboembolic complications, degeneration and infections.

Accordingly, consistent with the developing practice of the medical profession, there is a continuing need for improved replacement valves for use in a variety of positions within the natural heart as well as alternative locations in the circulatory system. Such replacement valves should incorporate lessons learned in clinical experience, particularly the need for reduction of stress on the valve leaflets and the maintenance of desirable structural and functional features. In particular, such replacement valves should restore, maintain and improve both mechanical and biological functions of the valve while avoiding the synergetic problems of current tissue engineered decellularized or bioresorbable tissues, such as immunogenicity, strong inflammatory response, anti-thrombogenicity, mechanical stability, and toxicity problems among others. Further, such replacement valves should be relatively simple and easy to manufacture in a consistent manner.

### SUMMARY

Directed at achieving the foregoing objective and to remedying the problems of traditional heart valve replacements, disclosed herein are preparation and use of a novel engineered human heart valve tissue, novel tissue heart valve constructions, and components thereof, and simplified methods of fabricating the same.

The tissue-engineered heart valve of the present invention is comprised of elements, such as leaflets, formed from self-supporting human engineered tissue. Such self-supporting tissue is comprised of living biological cells and extracellular matrix without the presence of nonviable exogenous scaffolding structures. This provides several theoretical advantages. First, a living valve implies responsive and self-renewing tissue with an inherent healing potential. Second, its biological matrix can be remodeled by the body according to the needs of the environment. Third, the absence of synthetic scaffolding tissue will preclude foreign body reaction, allow complete valve integration, and limit valve infection. Thus, the tissue-engineered heart valve of the present invention consists of totally living human tissue which could theoretically function like a native biological structure with the potential to grow, to repair and to remodel. The valve would remodel into a human living valve and adapt to its new environment, such as supporting human body's growth from infant to adult.

Generation of such self-supporting human engineered tissue is achieved by a “self-assembly” approach. The innovative self-assembly approach takes advantage of the abundant endogenous synthesis of extracellular matrix by cells, such as mesenchymal cells, when cultured in the presence of ascorbic acid. The resulting tissue structure displays histological organization, extracellular matrix composition, cell differentiation markers, and cellular functions observed in natural tissues. Advantageously over traditional tissue engineering approaches, the use of the self-assembly approach allows normal human cell-cell and cell-extracellular matrix interactions. In addition, it allows the secretion of important natural growth factors and cytokines, and the formation of a mature connective tissue desired for heart valves. Thus, the tissue-engineered heart valve of the present invention comprises cells that remain metabolically active and that undergo normal mitosis.

This strategy of restoring the natural biological functions of the valve is a paradigm shift in heart valve therapy: a new tissue-type valve that will remodel into recipient's own living valve. In order to provide longer term durability, the valve replacement should restore, maintain and improve both mechanical and biological functions of the valve. To accomplish this, the valve should be able to self-repair over years of implantation.

In some embodiments of the present invention, the human engineered tissue-type heart valve comprises a plurality of leaflets assembled to form a heart valve, wherein each leaflet is comprised of at least five layers of at least one living tissue sheet fused together to form a self-supporting human engineered tissue. It may be appreciated that each leaflet may be comprised of more than five layers, such as at least seven layers, at least nine layers, or more layers. Typically, the living tissue sheet is formed from an extracellular matrix secreted by mesenchymal cells. The mesenchymal cells may be, for example, allogeneic, autologous, genetically-modified or a combination of these. In some embodiments, the mesenchymal cells comprise dermal fibroblasts and adventitial fibroblasts. In other embodiments, the mesenchymal cells comprise myofibroblasts. And, in still other embodiments, the mesenchymal cells comprise interstitial valvular cells, endothelial cells or a combination of these. The living tissue sheet may alternatively be formed from an extracellular matrix secreted by embryonic, post-natal or adult stem cells. Similarly, the stem cells may be allogeneic, autologous, genetically-modified or a combination of these. In some embodiments, the at least one living tissue sheet includes collagen type I, collagen type III, elastin, glycosaminoglycans, growth factors, glycoproteins and water.

As mentioned, the layers may be portions of a single living tissue sheet, portions of several living tissue sheets, or both. For example, the at least five layers of at least one living tissue sheet may comprise at least five living tissue sheets stacked on top of each other. Or, the at least five layers of at least one living tissue sheet may comprise one living tissue sheets folded to create five layers. The resulting human engineered tissue generally has a thickness in the range of approximately 0.16 mm to 0.6 mm, preferably in the range of approximately 0.3 mm to 0.6 mm. Thus, in some embodiments, the human engineered tissue-type heart valve of the present invention comprises a plurality of leaflets assembled to form a heart valve, wherein each leaflet is comprised of layers of at least one living tissue sheet fused together to form a self-supporting human engineered tissue having a thickness of at least approximately 0.16 mm.

In other embodiments, the human engineered tissue-type heart valve of the present invention comprises a plurality of leaflets arranged to form a heart valve, wherein each leaflet is comprised of layers of at least one allogeneic living tissue sheet fused together to form a self-supporting human engineered tissue which undergoes living cell replacement upon implantation in a patient so that at least some of the allogeneic cells are replaced with the patient's living cells. In some instances, the majority of the allogenic cells are replaced with the patient's living cells. In other instances, approximately all of the allogenic cells are replaced with the patient's living cells. Further, the self-supporting human engineered tissue typically undergoes remodeling upon implantation in the patient.

The allogeneic cells may comprise mesenchymal cells. In some embodiments the mesenchymal cells comprise dermal fibroblasts or adventitial fibroblasts and in other embodiments the mesenchymal cells comprise interstitial valvular cells, myofibroblasts, endothelial cells or a combination of any of these. The allogeneic cells may comprise embryonic, post-natal or adult stem cells.

As in the previously described embodiments, each leaflet may be comprised of at least five layers. And, each leaflet may have a thickness in the range of approximately 0.15 mm to 0.6 mm, preferably in the range of approximately 0.3 mm to 0.6 mm.

The present invention further sets forth methods of making human engineered heart valves. In one embodiment, the method comprises generating at least one living tissue sheet by secreting an extracellular matrix from cells, layering the at least one living tissue sheet to form a layered construct having at least seven layers, and culturing the layered construct to fuse the layers to form a human engineered tissue.

Layering the at least one living tissue sheet may comprise stacking a plurality of individual sheets on top of each other. Alternatively or in addition, layering may comprise folding a single sheet upon itself. Such layering of the at least one living tissue sheet may comprise creating enough layers so that the human engineered tissue has a thickness in the range of approximately 0.16 mm to 0.6 mm, preferably in the range of approximately 0.3 mm to 0.6 mm.

The culturing step may comprise exposing the layered construct to L-ascorbate acid or a phosphate derivative of L-ascorbate acid serum. Optionally, the culturing step may also comprise anchoring the layered construct to reduce shrinkage. In addition, the forming step may comprise cutting each leaflet shape out of the human engineered tissue.

Further, the cells may comprise mesenchymal cells, or the cells may comprise embryonic, post-natal or adult stem cells.

The present invention further sets forth methods of preparing human engineered tissue for use in making a heart valve. In one embodiment, the method comprises generating at least one living tissue sheet by secreting an extracellular matrix from cells, layering the at least one living tissue sheet to form a layered construct, culturing the layered construct to fuse the layers to form the human engineered tissue, and regulating shrinkage of the human engineered tissue. In some embodiments, regulating shrinkage comprises anchoring the human engineered tissue. Anchoring may comprise placing a plurality of anchors upon the human engineered tissue. Anchors may be placed in a generally rectangular shape or in a generally circular or oval shape, to name a few. In other embodiments, regulating shrinkage comprises maintaining the human engineered tissue in wet conditions. Such wet conditions may comprise wet with HEPES, high glucose and Dulbecco Modified Eagle Medium. In still other embodiments, regulating shrinkage comprises creating a surface adhesion on the human engineered tissue to reduce shrinkage.

In another embodiment, the method comprises generating at least one living tissue sheet by secreting an extracellular matrix from cells, layering the at least one living tissue sheet to form a layered construct, culturing the layered construct to fuse the layers to form the human engineered tissue, and cutting a leaflet shape out of the human engineered tissue which is dimensionally larger than a desired leaflet shape to account for shrinkage. For example, cutting each leaflet shape may comprise punch cutting with a die having the leaflet shape. Or, cutting each leaflet shape may comprise cutting around a template having the leaflet shape. In some embodiments, dimensionally larger is approximately 50 percent larger. Such a method may further comprise constructing a heart valve using the leaflet shape.

The present invention does not limit its scope by using one particular technique sequence in the preparation of the cardiac valve. It is implicit that different sequences could be used to form a wide variety of valvular sizes and shapes to satisfy functionality of a tissue-engineered living human valve. Further, each portion of the valve may be fabricated from a tissue-engineered tissue generated by a different technique or having different characteristics. For example, one leaflet may be cut from a planar tissue formed using a single step folding technique and another leaflet may be cut from a tubular tissue produced using a rolling technique. The leaflets can then be combined in the assembly of a single valve.

Overall, an exemplary tissue-engineered living human valve includes a plurality of tissue leaflets which are cut from a mature human tissue-engineered tissue. The leaflets are attached together to form a dimensionally stable and consistent coapting leaflet subassembly when subjected to physiological pressures. Then each of the leaflets of the subassembly is aligned with and individually sewn to a wireform, typically from the tip of one wireform commissure, uniformly around the leaflet cusp perimeter, to the tip of an adjacent wireform commissure. The wireform is usually covered with human tissue-engineered tissue but can alternatively be covered with cloth. The sewed sutures act like similarly aligned staples, all of which equally take toe loading force acting along the entire cusp of each of the pre-aligned leaflets. The resulting tissue-wireform structural assembly thereby formed reduces stress and potential fatigue at the leaflet suture interface by distributing stress evenly over the entire leaflet cusp from commissure to commissure.

Thus, the present invention provides a human engineered tissue-type heart valve comprising a tissue leaflet subassembly mated with a wireform to form a heart valve, wherein each leaflet is comprised of at least five layers of at least one living tissue sheet fused together to form a self-supporting human engineered tissue, and wherein at least a portion of the wireform is covered with the tissue. In some embodiments, the heart valve further comprises a support stent mated with the wireform and at least a portion of the support stent may be covered with the tissue. And, in some embodiments, the heart valve further comprises an adaptable structural interface attached to the support stent and at least a portion of the adaptable structural interface may be covered with the tissue.

Other objects and advantages of the present invention will become apparent from the detailed description to follow, together with the accompanying drawings. All publications, figures, patents and patent applications cited herein are hereby expressly incorporated by reference for all purposes to the same extent as if each was so individually denoted.

### BRIEF DESCRIPTION OF THE DRAWINGS

FIG. 1 is a schematic illustration of obtaining cells from a patient;

FIG. 2 illustrates plating of the cells in petri dishes so that a living tissue sheet is formed in each dish;

FIG. 3 illustrates detachment of the living tissue sheet from the dish with the use of forceps;

FIG. 4 is a perspective view of living tissue sheets stacked so the sheets are directly superimposed;

FIG. 5 schematically illustrates overlapping of living tissue sheets to create a circular layered tissue construct;

FIG. 6 illustrates the stacking of irregularly shaped living tissue sheets to form a regularly shaped tissue construct;

FIG. 7 illustrates the stacking of sheets with alternating orientations;

FIG. 8 is a perspective view illustrating the folding of a sheet upon itself in an accordion-type fashion;

FIG. 9 is a perspective view illustrating the folding of a sheet in repeated halves;

FIG. 9A is a perspective view illustrating a wrapping technique;

FIG. 10 is a histological view of a mature-like human engineered tissue of the present invention;

FIG. 11 is a perspective view of a tissue with a weighted device applying pressure normal to the plane of the tissue;

FIGS. 11A-11B illustrate fiber orientation in relation to anchor placement;

FIG. 12 illustrates anchor placement along the entire periphery of the tissue construct;

FIGS. 13, 13A, 14, 14A illustrate the creation of specific collagen fiber orientation to coordinate with construct geometry;

FIGS. 15A-15B illustrate optional preparation for delivery of tissue construct;

FIG. 16 is a perspective view illustrating the step of templating and trimming exemplary leaflets used in making a tissue heart valve of the present invention;

FIG. 17 is a top view of a tissue having cutouts wherein tissue was die or template cut and removed;

FIG. 17A illustrates the leaflets cut from the tissue of FIG. 17;

FIG. 18 is a top view of an embodiment of a living tissue-engineered human cardiac valve of the present invention;

FIG. 19 is a perspective view of the embodiment illustrated in FIG. 18;

FIG. 20 is an exploded perspective view of an exemplary heart valve of the present invention illustrating the assembly relationship of the standardized components and alternative valve attachment application structures;

FIG. 21 illustrates the initial steps of templating and pre-aligning the leaflets of the valve subassembly;

FIG. 22 shows additional steps in the pre-alignment of the valve leaflet subassembly;

FIG. 23 is an enlarged view illustrating an exemplary attachment step of the pre-aligned leaflets to a wireform commissure tip;

FIG. 24 is a perspective view illustrating the subsequent preliminary attachment of the exemplary leaflet cusps to the wireform of FIG. 23;

FIG. 25 is a perspective view illustrating the uniform attachment of the perimeter cusps of leaflet to the cloth, or human engineered heart valve tissue covered wireform;

FIG. 26 is an enlarged view of one of the pairs of attached leaflet tabs of FIG. 25 illustrating the uniform attachment of the cusps to the wireform commissure tip;

FIG. 27 is a perspective view illustrating the attachment of the exemplary tissue leaflet-wireform structural subassembly to an exemplary stent of the present invention;

FIG. 28 is an enlarged view of one of the pairs of leaflet tabs of FIG. 27 illustrating a further attachment step of the stent to the wireform at the commissure tip, clamping the leaflet cusps therebetween;

FIG. 29 is an enlarged view of one of the commissure tips of the tissue-wireform structural assembly of FIG. 28 illustrating the clamping of the leaflets by the stent;

FIG. 30 is a perspective view illustrating a final attachment step of the exemplary tissue-wireform structural assembly to the stent;

FIG. 31 is an enlarged view taken on circle 13 of FIG. 30 illustrating additional exemplary attachment techniques;

FIG. 32 is an enlarged view taken on circle 14 of FIG. 30 illustrating additional exemplary attachment techniques;

FIG. 33 is a perspective view illustrating an exemplary attachment step of the tissue leaflet tabs at the commissure tip;

FIG. 34 is a view similar to FIG. 33 illustrating an alternative attachment step;

FIG. 35 is an exploded perspective view illustrating an exemplary multi-piece stent formed of a flexible support and an associated stiffener of the present invention;

FIG. 36 is a perspective view illustrating the attachment of the support to the stiffener of FIG. 17;

FIG. 37 is a perspective view illustrating an initial step in the covering of the stent components of FIG. 36 with cloth, or human engineered heart valve tissue;

FIG. 38 is an enlarged view of the top of FIG. 37 illustrating additional steps in the attachment of the cloth, or human engineered heart valve tissue to the stent components;

FIG. 39 is a perspective view illustrating additional steps of fabricating sewing tabs for attaching the cloth, or human engineered heart valve tissue to the stent components;

FIG. 40 is an enlarged view of a portion of FIG. 38 illustrating subsequent fabrication steps;

FIG. 41 is an enlarged cross-sectional view taken on line 23-23 of FIG. 40;

FIG. 42 is a view similar to FIG. 40 illustrating additional fabrication steps;

FIG. 43 is a perspective view of the cloth-covered, or human engineered heart valve tissue-covered stent of FIG. 36 illustrating the cloth, or tissue seating lip;

FIG. 44 is an enlarged cross-sectional view on line 26-26 of FIG. 43 illustrating additional aspects of the fabrication of the exemplary stent assembly;

FIG. 45 is a perspective view illustrating initial components of an exemplary suture ring of the present invention;

FIG. 46 is an enlarged cross-sectional view illustrating aspects of the fabrication of the exemplary suture ring; FIG. 47 is a perspective view illustrating additional features of the exemplary suture ring assembly;

FIG. 48 is an enlarged sectional view of a portion of FIG. 47 illustrating additional aspects of the fabrication of the suture ring assembly;

FIG. 49 is an enlarged sectional view illustrating additional aspects of the finished exemplary suture ring assembly;

FIG. 50 is an exploded perspective view illustrating positioning and assembly of a suture ring and leaflet subassembly configuration;

FIG. 51 is a top perspective view illustrating additional suture ring leaflet subassembly attachment steps;

FIG. 52 is a bottom perspective view illustrating further exemplary suture ring attachment steps;

FIG. 53 is a cutaway perspective view illustrating an exemplary attachment of an outflow conduit to an exemplary valve of the present invention;

FIG. 54 is an enlarged cross-sectional view illustrating additional aspects of the conduit attachment;

FIG. 55 is a cross sectional view similar to FIG. 54 illustrating alternative conduit attachment features; and

FIG. 56 is an exploded perspective view illustrating additional valve attachment alternatives of the present invention.

FIG. 57 is a perspective view of an additional embodiment of a living tissue-engineered human cardiac valve of the present invention;

FIG. 58 illustrates the use of the human engineered tissue to cover a wireform.

### DETAILED DESCRIPTION

The present invention provides human-engineered tissue heart valves which replicate both mechanical and biological valvular functions in vitro. Importantly, the human-engineered heart valves are comprised of tissue cells which are used to form, to maintain and to improve morphologically and histologically mature valvular components.

The subsections below describe preparation and use of human engineered tissue for producing living tissue-engineered human valves in vitro.

Cell Source

A variety of cells can be used in the human-engineered tissue of the present invention. Preferred cell types include embryonic stem cells, post-natal stem cells, adult stem cells, mesenchymal cells, especially fibroblasts, interstitial cells, endothelial cells, smooth or skeletal muscle cells, myocytes (muscle stem cells), chrondocytes, adipocytes, fibromyoblasts, and ectodermal cells, including ductile and skill cells, hepatocytes, Islet cells, cells present in the intestine and other parenchymal cells, osteoblasts and other cells forming bone or cartilage. In some cases it may also be desirable to include nerve cells.

Cells can be normal or genetically engineered to provide additional or normal function. Methods for genetically engineering cells with retroviral vectors, polyethylene glycol, and other methods known to those skilled in the art can be used.

Cells may be autologous, allogeneic or xenogeneic, however autologous or allogeneic cells are preferred. Immunologically inert cells, such as embryonic or fetal cells, stem cells, and cells genetically engineered to avoid the need for immunosuppression may also be used. Methods and drugs for immunosuppression are known to those skilled in the art of transplantation.

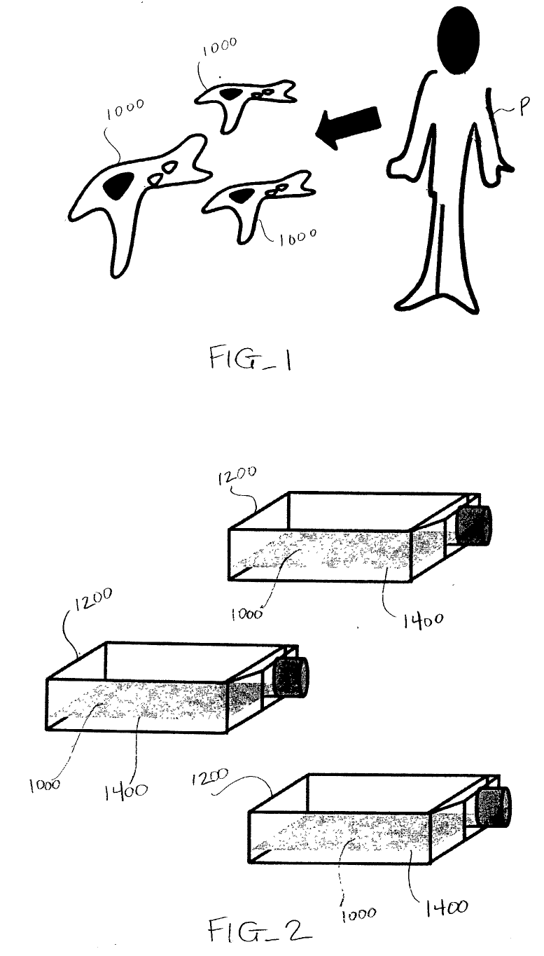

In some embodiments, cells are obtained by biopsy and dissociated using standard techniques, such as digestion with a collagenase, trypsin or other protease solution. FIG. 1 illustrates obtaining cells 1000 from a patient P. In some embodiments, the dermal layer of a skin biopsy is harvested and digested with collagenase according to the method of Germain and Auger, “Tissue engineered biomaterials: biological and mechanical characteristics”, In: Wise, Trantolo, et al. editors: “Encyclopedic handbook of biomaterials and bioengineering”, NY, N.Y.: Marcel Dekker Inc., 1995, pp.699-734. Briefly, cells are harvested following centrifugation of the digested dermal fragments, and expanded in cell culture media. All cell cultures are used between their fourth and eight passages, and kept incubated at 37° C. and 8% CO₂. Cells can be easily obtained through a biopsy anywhere in the body, for example, skeletal muscle biopsies can be obtained easily from the arm, forearm, or lower extremities, and smooth muscle can be obtained from the area adjacent to the subcutaneous tissue throughout the body. The biopsy can be effortlessly obtained with the use of a biopsy needle, a rapid action needle which makes the procedure extremely simple and almost painless. Cells may also be procured from, for example, blood vessels, valves and discarded tissues, such as foreskins.

In preferred embodiments, mesenchymal cells are used. Native heart valves are populated with mesenchymal cells, such as endothelial cells and interstitial cells. Among these populations, there exist a considerable phenotypic heterogenicity. It has also been suggested, such as by Beresford, “Transdifferentiation and the vascular wall”, In: Zilla and Greisler, editors: “Tissue engineering of vascular prosthetic grafts”, Austin, Tex.: R G Landes, 1999, pp.403-16, that embryonic endothelial cells trans-differentiate into valvular interstitial cells into the extracellular matrix during the postnatal valve development. More importantly, phenotypes of those mesenchymal cells are modulated through environmental stimuli such as complex mechanical forces of valve when closing and opening, as described by Schoen and Levy, “Tissue heart valves: current challenges and future research perspectives”, Journal of Biomedical Material Research, Vol. 47, 1999, pp.439-465.

In particular, fibroblasts, such as dermal fibroblasts or adventitial fibroblasts, may be used. Fibroblasts are easily available, and they are the primary collagen secreting cells in connective tissues. Dermal fibroblasts are typically harvested from normal adult skin specimen removed during reductive breast surgery, or from neonatal foreskin. The potential of human fibroblasts for cardiovascular application is enormous for both allogeneic and autologous grafts since cells contained in one square-inch of foreskin can be used to grow many feet of tissue.

Preparation of Human Sheets of Living Tissues

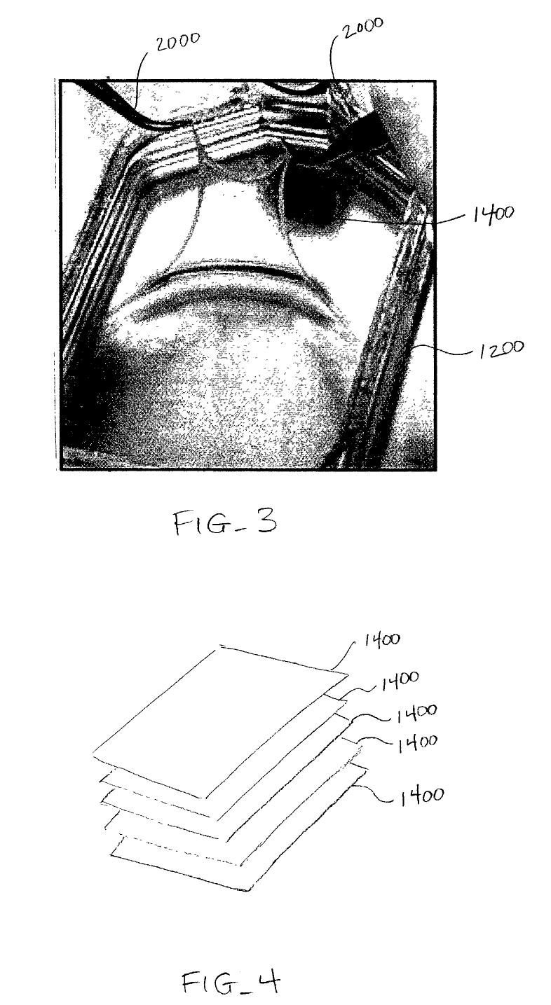

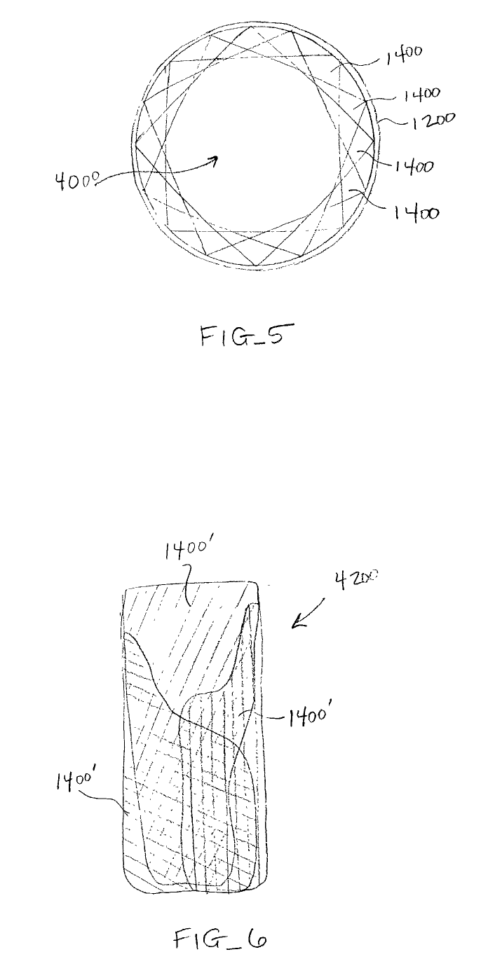

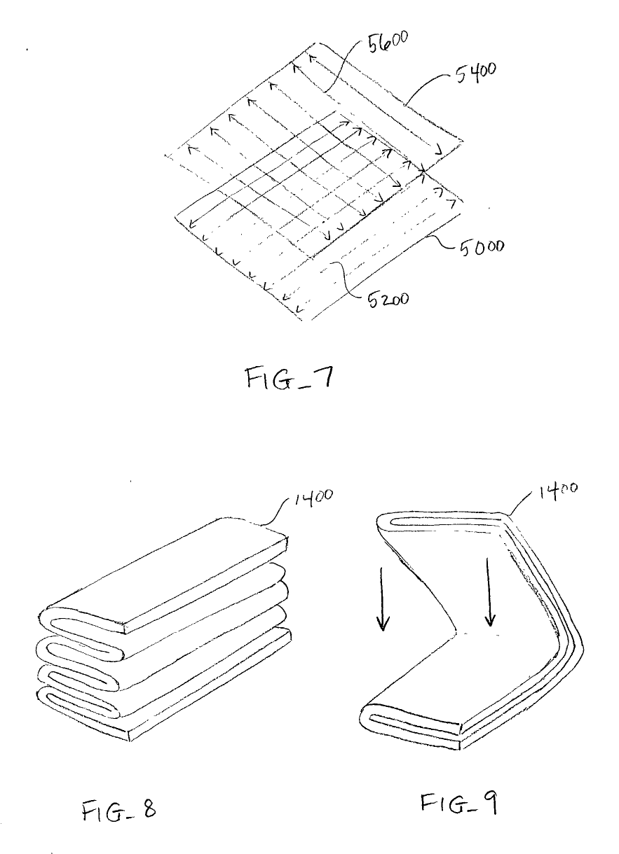

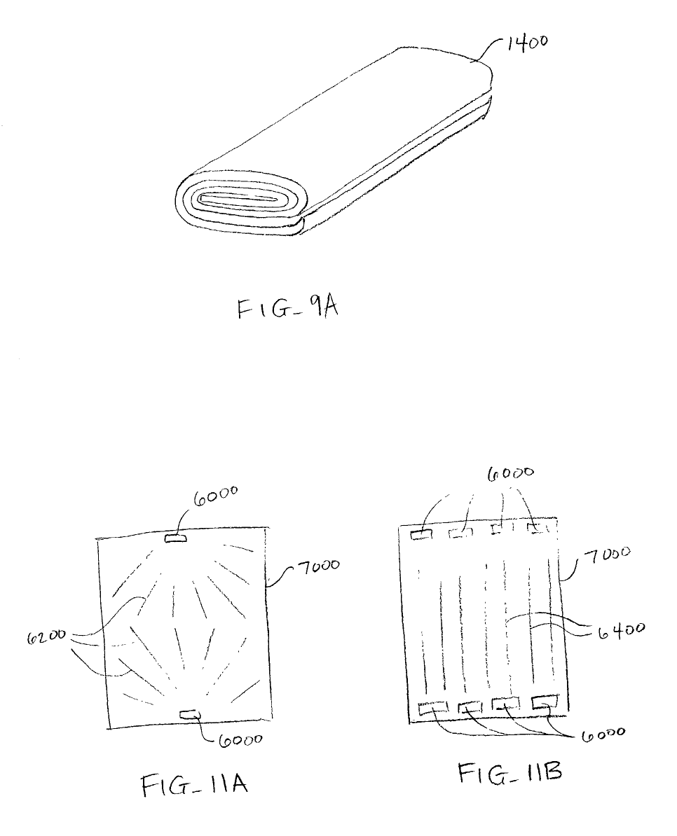

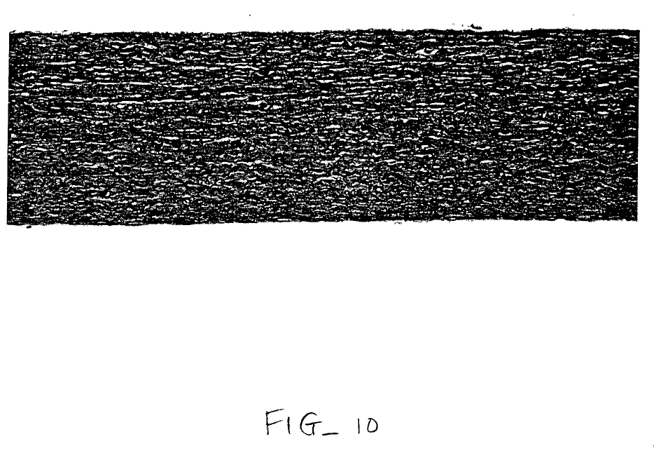

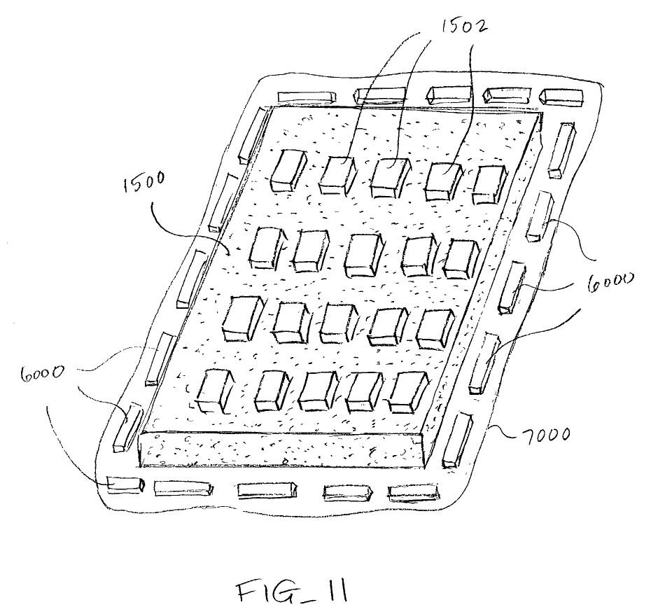

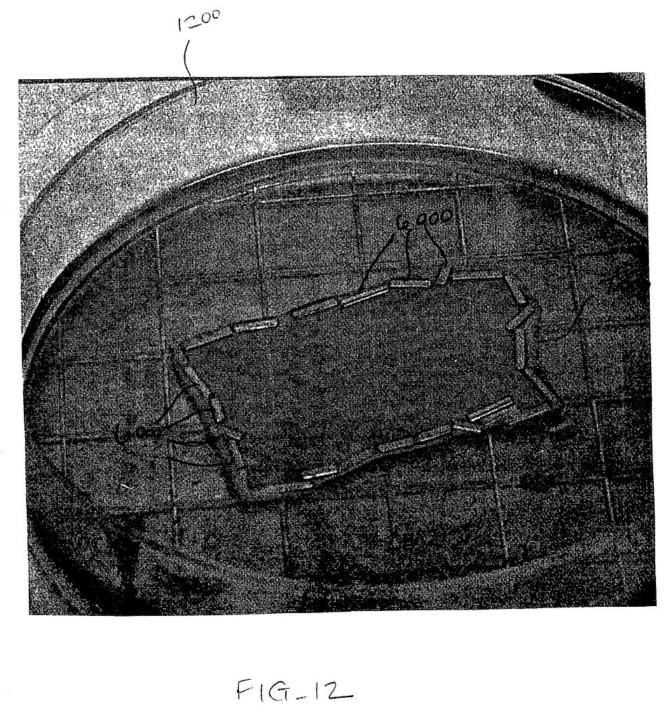

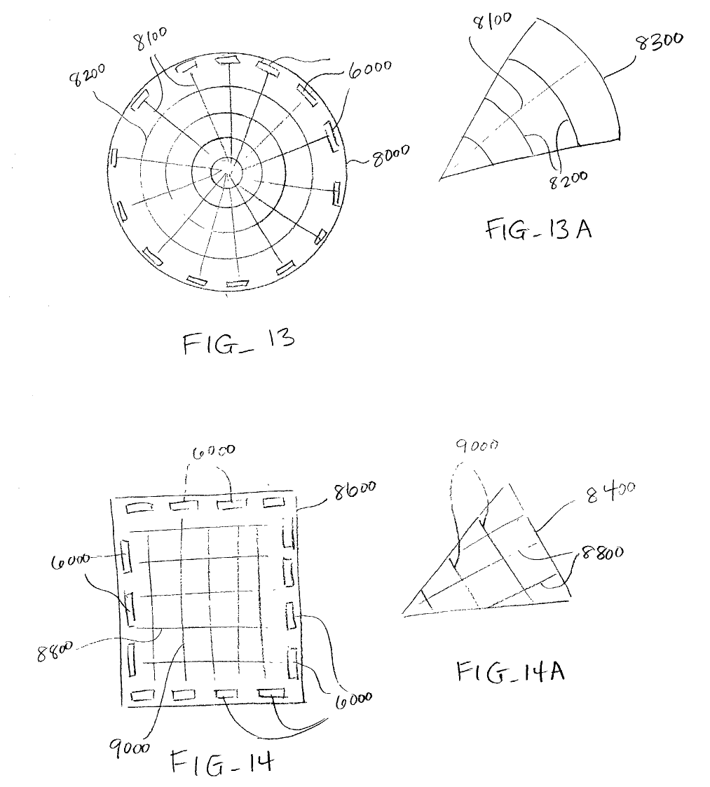

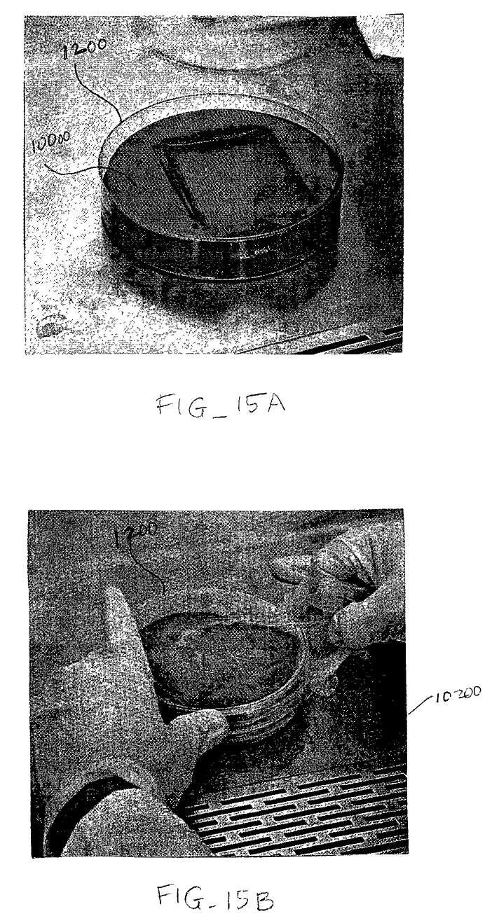

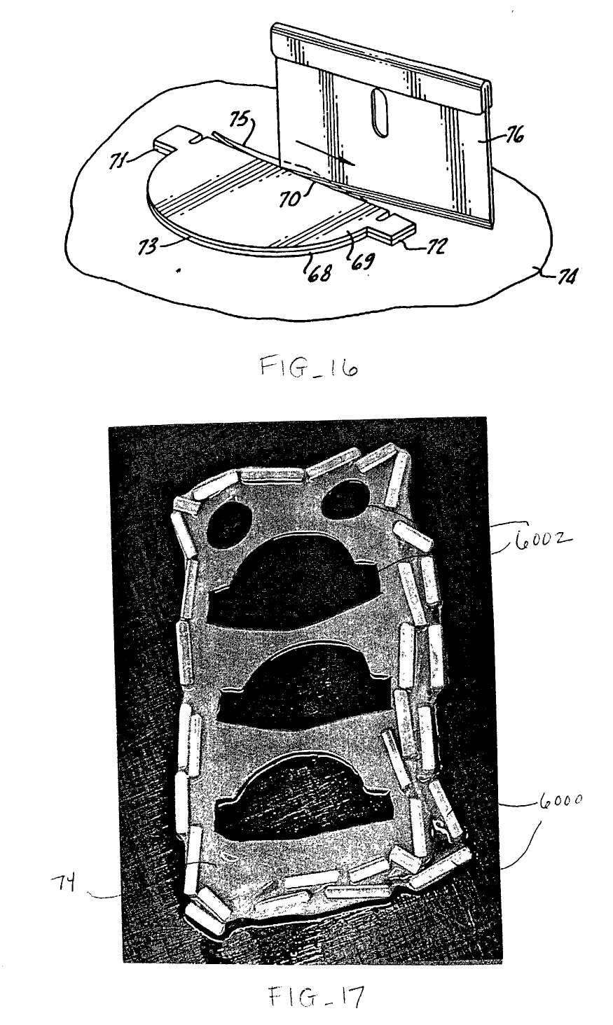

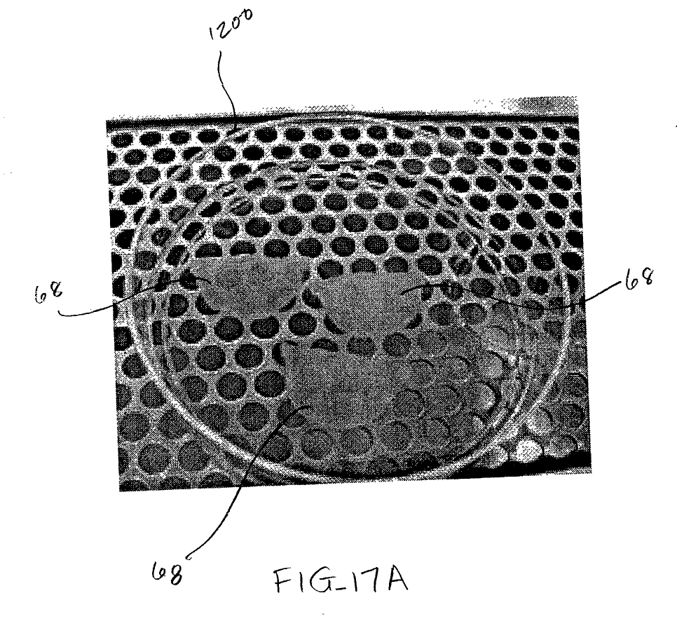

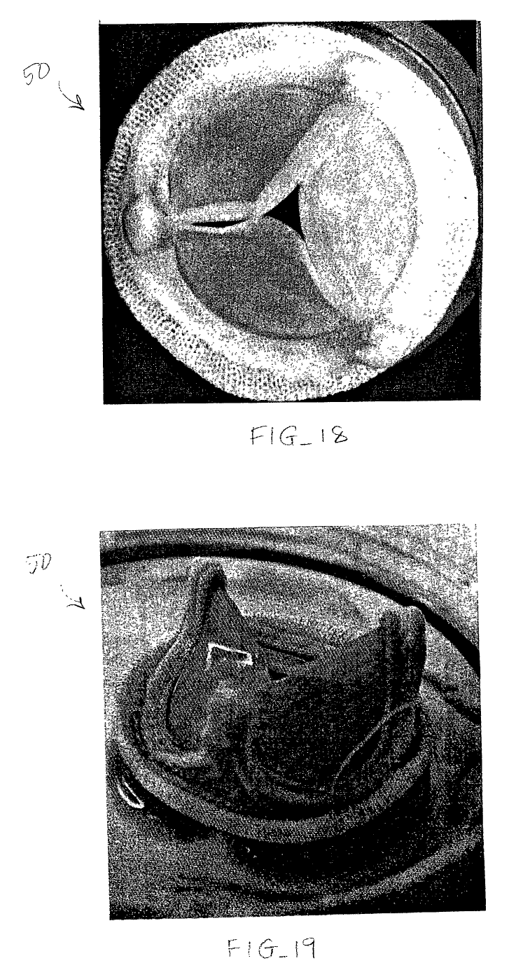

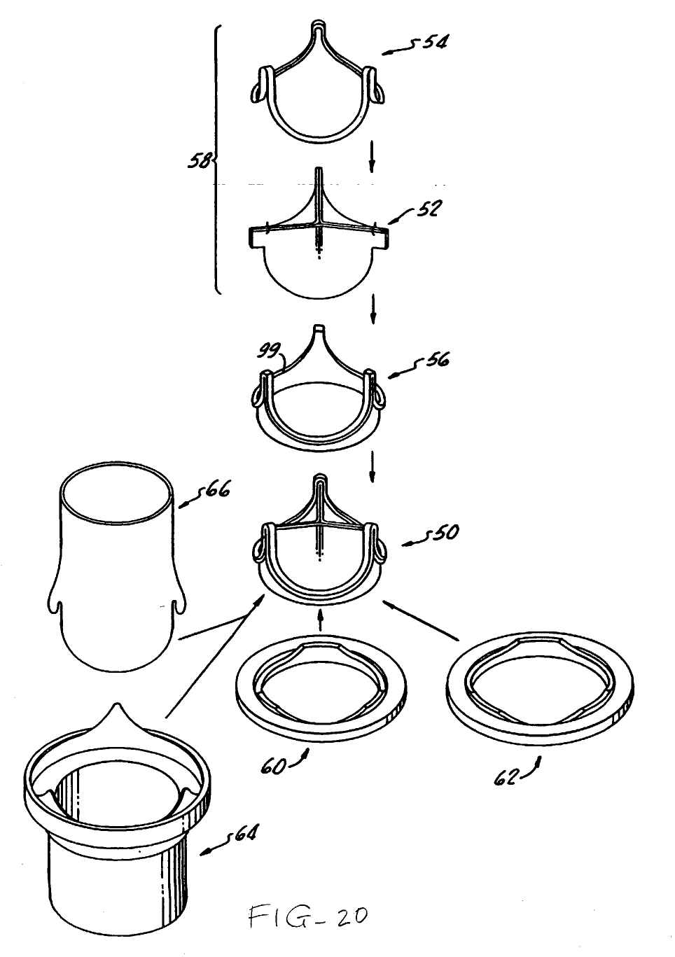

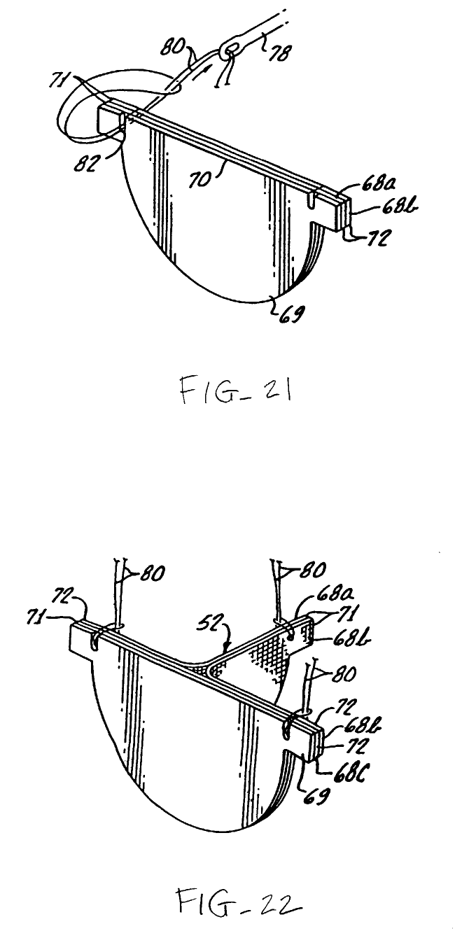

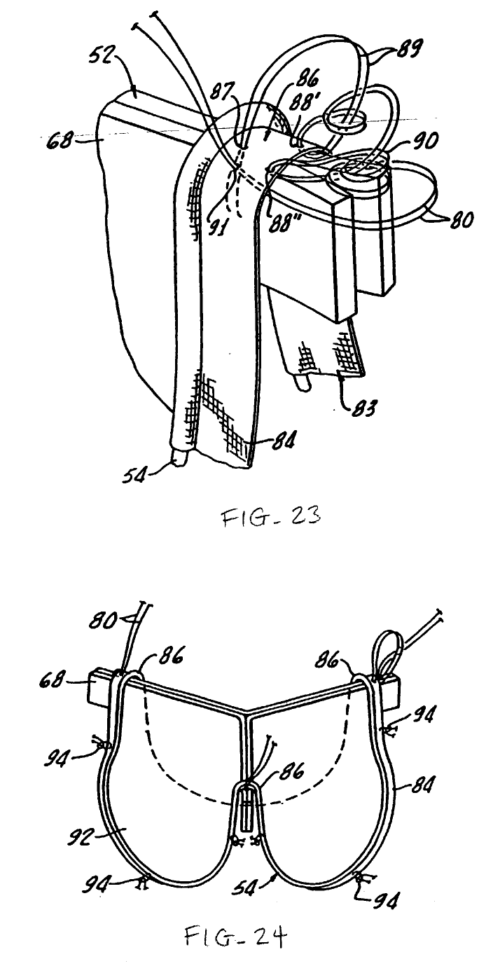

The human-engineered tissue used to create the heart valves of the present invention is formed from at least one sheet of living tissue. As illustrated in FIG. 2, the cells 1000 are plated in sterile petri dishes 1200 so that a living tissue sheet 1400 is formed in each dish 1200. Each living tissue sheet 1400 is comprised of an endogenous extracellular matrix to which additional cells become attached. The extracellular matrix is secreted by cells 1000, such as mesenchymal cells, embryonic stem cells, or adult stem cells, to name a few. When mesenchymal cells, such as dermal fibroblasts, are cultured in a planar culture substratum using L-ascorbate acid or a phosphate derivative of L-ascorbate acid (e.g. Asc 2-P), serum, and growth factors, they show an abundant synthesis of extracellular matrix proteins. This creates the basis of the endogenous extracellular matrix. L-ascorbic acid plays an important role since it is a cofactor for the hydroxylation of proline and lysine residues in collagen, as described in Hata, Ryu-Ichiro et al., “L-Ascorbic Acid 2-Phosphate Stimulates Collagen Accumulation, Cell Proliferation, and Formation of a Three-Dimensional Tissuelike Substance by Skin Fibroblasts”, Journal of Cellular Physiology, 138:8-16(1989), and also it increases both the rate of transcription of procollagen genes and stability of procollagen mRNA. The extracellular material is comprised of different proteins, such as essentially collagen type I, other collagen types, elastin, glycosaminoglycans, growth factors, and glycoproteins, to name a few. The resulting living tissue formed from the extracellular matrix is called a ‘living tissue sheet’.

An exemplary embodiment of methodology for generating such living tissue sheets 1400 is described in U.S. Pat. No. 5,618,718 to Auger et al., incorporated herein by reference for all purposes. In summary, Auger et al. describes that dermal fibroblasts, at a concentration equivalent to 10⁴ cells/cm², are plated into 75 cm² sterile Petri dishes. Cell medium is supplemented with a 3:1 DMEM and Ham's F12 modified medium, fetal bovine serum, penicillin and gentamicin, and with an ascorbic acid solution (50-100 μg/ml) every day. Culture conditions are kept at 92% air and 8% CO₂ at full humidity. Culture time is approximately three weeks. At the end of the maturation time, the sheet of living tissue 1400 spontaneously detaches from the substratum. FIG. 3 illustrates detachment of the living tissue sheet 1400 from the dish 1200 while the sheet 1400 is held by forceps 2000.

It can be appreciated that a variety of methods can be used to prepare the sheets of living tissue, e.g. U.S. Pat. No. 5,618,718 (Auger et al.); Ye, Qing et al., “Tissue engineering in cardiovascular surgery: new approach to develop completely human autologous tissue”, European Journal of Cardio-Thoracic Surgery, 17 (2000) 449-454; L'Heureux, Nicolas et al., “A completely biological tissue-engineered human blood vessel”, The FASEB Journal, Vol 12, January 1998, pp. 47-56; Michel M. et al., “Characterization of a new tissue-engineered human skin equivalent with hair.” In Vitro Cell Dev Biol Anim. June 1999;35(6):318-26; Pouliot R. et al., “Reconstructed human skin produced in vitro and grafted on athymic mice”, Transplantation Jun. 15, 2002;73(11):1751-7, all of which are incorporated herein by reference for all purposes.

In addition, the present invention is not limited in scope by using one particular cell type, origin, age, maturation time, component concentration, and culture conditions to generate the sheet of living tissue 1400. Further, the present invention is not limited in scope by producing any particular shape (i.e. thickness and size) of living tissue sheet 1400.

Preparation of Engineered Valve Tissue

The human-engineered valve tissue of the present invention is formed from superimposing a plurality of individual living tissue sheets 1400. As described above, the living tissue sheets 1400 are comprised of an extracellular matrix secreted by cells 1000, such as mesenchymal cells. The extracellular matrix is produced with many in vivo-like properties including supermolecular organization of collagen. Collagen is not only processed essentially complete, but is also cross-linked efficiently and the collagen fibrils are assembled into bundles. When the sheet 1400 is layered upon itself, for example by folding or wrapping, or a plurality of sheets 1400 are stacked or superimposed, a three-dimensional construct having desired structural characteristics is formed in culture.

In some embodiments, the sheets of living tissue are stacked in a cell culture dish, either directly superimposed or in an overlapping fashion. FIG. 4 illustrates five individual living tissue sheets 1400 directly superimposed in a stacked formation. Alternatively, by overlapping tissues a variety of shapes may be formed. For example, as schematically illustrated in FIG. 5, rectangular sheets of living tissue may be arranged in a petri dish 1200 in an overlapping fashion to create a circular layered tissue construct 4000. Or, as illustrated in FIG. 6, irregularly shaped living tissue sheets 1400′ may be stacked in a manner to form a regularly shaped tissue 4200. In addition, the individual sheets may be stacked in the same orientation or the orientation of the sheets may be varied to create specific effects in the resulting tissue. FIG. 7 illustrates the stacking of sheets 1400 having orientations designated by arrows. Here, a first sheet 5000 is shown having a first orientation 5200. A second sheet 5400 is shown having a second orientation 5600. The sheets 5000, 5400 are stacked so that the orientations 5200, 5600 are perpendicular to each other. This may provide a different effect than if the sheets were stacked so that the orientations 5200, 5600 were parallel to each other or in any other relation.

Alternatively or in addition, one or more living tissue sheets can be folded to form a multitude of layers. For example, as illustrated in FIG. 8, a sheet 1400 may be folded upon itself in an accordion-type fashion to form a multitude of layers. Or, as shown in FIG. 9, a sheet 1400 may be folded in repeated halves to superimpose portions of the sheet upon itself. Similarly, two or more sheets 1400 may be stacked and then folded upon itself to create even more variety of layering. Alternatively or in addition, the layering technique could also be extended to the use of wrapping techniques, such as wrapping a sheet 1400 around itself in the style of a cinnamon roll as illustrated in FIG. 9A. Again, one or more sheets may be stacked and then wrapped or any combination of layering techniques.

When layering, the living tissue sheets are held together by adhesion or surface adhesion between the sheets. Any number of living tissue sheets may be used, preferably five or more, more preferably seven or more, and more preferably, nine, ten or eleven or more. The sheets are delicately handled with forceps and superimposed or otherwise assembled to form the human engineered tissue construct. By maintaining this construct in culture medium supplemented with ascorbic acid under conditions similar to those described in U.S. Pat. No. 5,928,281, incorporated by reference for all purposes, the living tissue sheets will fuse together to form a mature-like human engineered tissue, as illustrated in FIG. 10. FIG. 10 is a microscopic view of an embodiment of the tissue after maturation of nine sheets of living tissue containing fibroblasts and extracellular matrix constituents. This light microscopy demonstrates a tissue construct resembling that of a native tissue with dense extracellular matrix. In addition, the nine superimposed sheets of living tissue have fused together to form one single construct. Maturation time of the construct is dictated by the specific mechanical properties desired. In some embodiments, particularly wherein the tissue construct is comprised of nine layers of living tissue sheets, the maturation time is seven weeks. It has been found that mechanical strength of such a tissue plateaus after seven weeks of maturation.

Generally, the engineered tissue is thin enough to allow oxygen delivery through its surfaces to maintain metabolic needs yet thick enough to provide adequate strength and durability for use in heart valves. The current embodiments of the engineered valve tissue of the present invention are avascular, wherein the tissue does not include a microvasculature to deliver oxygenated blood to the tissue. Therefore, the tissue relies on oxygen diffusion from its surfaces to sustain the tissue. Due to oxygen diffusion limitations, the tissue thickness is currently an important consideration. In some embodiments of the present invention, the engineered tissue has a thickness ranging from approximately 0.1 mm, preferably 0.3 mm, for normal sized valves, to approximately 0.6 mm, for larger valves. The tissue preparation is suitable for the smallest valve sizes (known as size 19 mm) as well as larger sizes (known as size 33 mm).

As mentioned, it is from this human engineered tissue that the cardiac valves of the present invention will later be formed. It is desired that the engineered tissue be sufficiently strong and pliable for use in cardiac valves which provide complex mechanical and biological functions. It is also desired that the engineered tissue contains an optimized fibrous microstructure to resist stretching when subjected to supra-physiological blood pressures. Valve cusps made from this engineered tissue show adequate pliability to allow low pressure gradient throughout the opening of the valve and can withstand high aortic physiological pressure during closing. It is also desired that the tissue is also capable of promoting endogenous growth, self-repair for long-term structural integrity, and repair of damaged or diseased tissues.

The sheets are delicately handled with forceps and superimposed or otherwise assembled to form the human engineered tissue construct, as described above. The construct is maintained in culture medium supplemented with ascorbic acid so that the living tissue sheets fuse together to form a mature-like human engineered tissue. To keep the tissue fully immersed in the culture medium and to reduce shrinkage of the tissue during maturation, anchors may be applied to the maturing tissue. Anchors may include, for example, weights, ingots or bars which rest on the surface of the tissue. Such anchors may be made from any material that does not interfere with the development or differentiation of cells in the sheet of living tissue, such as stainless steel. Magnets or metal ingots coated in teflon or any polymer material known in the art to be compatible with tissue culture may also be used. Suitable weight values for the anchors for use with a tissue type can be determined empirically. Preferably, weights are chosen so that cell orientation and/or differentiation are induced but cell apoptosis is substantially avoided. Without the use of such anchors, the tissue would be free-floating, allowing cell traction forces to remodel the microstructure of fresh tissue leading to systematic shrinkage and the production of an irregular mass of scarring tissue. Tensile forces provided by the peripheral anchors overcome and prevent such tissue shrinkage.

In addition, application of a compressive force normal to the plane of the tissue construct may enhance fusion between adjacent layers of the tissue. Compression improves cell-cell contact between layers of tissue and encourages fusion of cells across layers of tissue. Such compression may be applied with the use of a weighted device applied to the tissue or superimposed planar sheets of tissue, thereby applying a force normal to the plane of the tissue. For example, as illustrated in FIG. 11, the weighted device may comprise a sponge 1500 upon which spaced apart weights 1502 are placed. The sponge 1500 is positioned upon the tissue construct 7000 so as to substantially cover and apply normal force to its planar surface. Optionally, the tissue construct 7000 may also be anchored around its perimeter with anchors 6000, as shown.

Preferably, the compressive force or pressure is applied evenly on the entire tissue surface. Therefore, it is preferable that a device adapted to the shape of the tissue be used to induce the fusion. The amount of pressure applied to the surface of the tissue stack can be adjusted according to the needs of the engineered tissue. Thus, in this example, the anchors 6000 may vary by weight and distribution to obtain the desired amount of pressure on the tissue. Of course, any other system using mechanic or hydraulic pressure could be used to provide this compression. The period of time for which this pressure has to be applied should be long enough to allow the complete fusion of the tissue layers, preferably 24 to 72 hours.

It is also preferable that the device used to apply pressure to the surface of the tissue be permeable to culture media in order to allow the nutrition of the living cells. The sponge 1500 shown in FIG. 11 is thus an acceptable way to generate such pressure as it is porous and permeable to culture media.

During the fusing process, mechanical stress may be used to induce cellular orientation and phenotypic modulation of the cells within the tissue. Thus, appropriate forces may be applied to maturating tissue in order to induce fiber orientation. Such forces may also prevent shrinkage and maintain the desired cell differentiation. Anchors may be used to maintain or create such desired cell differentiation and fiber orientation. The current invention provides a methods of anchoring maturing cultured tissues. In one embodiment, the method comprises an adjustable anchor means, preferably comprising a multiplicity of spaced apart anchors (such as moveable weights or ingots), wherein the anchors are suitable for (1) applying sufficient tension across the sheet of living tissue to prevent shrinkage and/or maintain cellular differentiation and/or induce orientation of cells in at least one sheet of living tissue and (2) allowing contraction of at least one sheet of living tissue once a predetermined threshold of tension is exceeded across the sheet of living tissue.

In this embodiment, the anchor-means are punctual, wherein each anchor-means holds the tissue substantially at a point in space. The anchor-means is also “adjustable” in that once the tissue has built up a tension higher than the maximum tension that can be held by the anchors (i.e. weights or ingots), the tissue can spontaneously contract and the anchors will be pulled along with the contracting tissue. Thus, the tension across the tissue cannot continue to build up when an adjustable anchor means as described is employed. The maximum tension that can build up across the tissue can be controlled by choosing suitable anchors (for example weights or ingots of a certain weight and number, or an adjustable frame that is designed to move in response to a certain tension or force). Since the anchors are moveable they can easily be placed on a sheet of tissue or removed therefrom. Thus, it is possible to optimize the amount of tension for any given tissue, for example, to enhance viability of cells in the tissue.

As an example, illustrated in FIG. 11A, placement of two anchors 6000 on a tissue construct 7000 can lead to a cord-like orientation (signified by lines 6200) of the cells and matrix between these two anchors 6000. If anchors 6000 are grouped to form two lines of anchors 6000 along the tissue construct 7000, as illustrated in FIG. 11B, fibers will align (signified by lines 6400) parallel to the mechanical forces induced by the anchors 6000.

Further, if the anchors 6000 are positioned along the perimeter of a rectangular tissue construct 7000, as illustrated in FIG. 12, the fibers and the cells will be oriented in the two dimensions of the plane created by the anchors 6000. This orientation of cells and extracellular matrix may be beneficial for the fusion process and may also improve certain functional properties of the tissue. As illustrated, a multiplicity of spaced apart anchors may be used for applying mechanical force to tissue in a punctuated or discontinuous manner along the edge of the sheet of living tissue. If the anchors are arranged very close to each other or so as to contact each other, they may displace each other somewhat when the tissue contracts. The amount and direction of mechanical force applied to the tissue can be controlled by varying the number, weight and position of the anchors. Hence, it is possible to optimize or fine-tune the mechanical force conditions for any particular type of tissue.

It may be appreciated that a continuous anchor, such as a frame or a ring of glass microfiber that circumscribes or encircles the tissue, may alternatively be used to induce cellular orientation. The induction of cell orientation may occur because the continuous anchor mechanically restricts the spontaneous contraction of the maturing cultured tissue, thereby creating a mechanical stress or tension across the tissue that induces cell orientation. However, the use of continuous anchors may only be suitable for particular tissue types which do not create high levels of tension during maturation.

Combining cell traction forces with specific construct geometry can provide a specific collagen fiber orientation to better support valvular function of the later constructed valve, such as coaptation of the leaflets, reduced regurgitation, effective orifice area, and low pressure gradients, to name a few. For example, as illustrated in FIG. 13, a disk-shaped construct 8000 having anchors 6000 positioned around its circumference, would force radial 8100 and circumferential 8200 collagen fibril orientation during the maturation of the tissue construct 8000. FIG. 13A illustrates a wedge-shaped portion 8300 cut from the disk-shaped construct 8000 wherein some edges of the portion 8300 fall along the lines of collagen fibril orientation. This may more closely mimic the native valve, wherein the same circumferential orientation of main collagen fibers support larger tensile forces distributed to the leaflet. Alternatively, as illustrated in FIG. 14 and FIG. 14A, a wedge-shaped portion 8400 cut from a rectangular construct 8600 can provide short 8800 and long 9000 axes orientations to support different loading conditions.

Delivery Conditions

If it is desired to transport the matured tissue, the tissue may be prepared for delivery as follows. As illustrated in FIG. 15A, each tissue construct contained in its respective culture dish 1200 is covered with a solid agar based nutrient medium 10000, preferably 1% agar in Dulbecco's Modified Eagle Medium (D-MEM), with buffer to maintain physiological stable pH. Covering the tissue construct with the agar based nutrient medium 10000, such a solid gel, also has the advantage of maintaining the weighed anchors in place and prevents any movement of the tissue and/or folds of the tissue construct during transport. In addition, the cells are kept viable by diffusion of the nutriments from the gel. FIG. 15B illustrates covering the dish 12 with a cover 10200 for shipping. Preferably, these dishes 1200 shall not be frozen but preheated at approximately 37° C. before shipping. Preheated ‘hot packs’, preferably at approximately 37° C., combined with insulated package, such as a standard cooler, may be used during transport. This delivery system allows maintenance of integrity and viability of the tissue for at least 24 hours.

Preconditioning

Once the tissue had matured and is released from its anchoring aids (e.g. stainless steel bars, or strings) using sterilized forceps in a controlled area, such as laminar flow hood, residual shrinkage may occur. To reduce such shrinkage, various preconditioning methods may optionally be undertaken. To begin, the tissue construct may be discharged from its culture medium, but kept wet, and be flattened at the bottom of another culture dish under a laminar flow hood for approximately fifteen minutes. During that period the shrinkage of the construct will settle.

Alternatively or in addition, other preconditioning methods can be used to adjust elasticity of the human tissue-engineered tissue before assembly of the valve or prior to implantation. Such preconditioning can include stretching or bursting the tissue to bigger dimensions than the dimensions from which the tissue was originally formed. One method of preconditioning the construct is by stretching or applying a given load repeatedly at a physiological frequency, such as applying a load ten times at a frequency of one Hertz. Such preconditioning produces a load-conditioned construct with reduced strain. Alternatively, the construct can be preconditioned for all dimensions, which can be achieved by a bursting pressure technique or circulatory fluid flow.

Cutting of Leaflets

The leaflets and other portions of the valve are cut from the tissue-engineered tissue by any suitable method. Preferred methods include template cutting and die cutting. In template cutting, templates are created for each portion of the valve wherein each template has an appropriate shape and size for the given portion. The template is placed on the tissue and a cutting blade is moved along the edge of the template to cut the underlying tissue into the same shape as the template. FIG. 16 illustrates a leaflet 68 being trimmed to a desired shape and size for the intended valve use using a template 69, defining a generally straight or linear coapting mating edge 70 having opposing ends 71, 72 and a generally arcuate peripheral cusp 73 extending therebetween. The leaflet 68 is placed on a cutting board 74 and the selected template 69 is then placed over the leaflet 68. Tissue 75 extending beyond the boundaries of template 69 is then cut away using a sharp razor blade 76 or similar cutting tool. Similarly, such portions may be die cut or punch cut from the tissue. A die is formed in the desired size and shape having a cutting edge along its periphery. The die is then placed on the tissue and pressed until the cutting edge cuts through the tissue. FIG. 17 illustrates tissue 74, supported by anchors 6000, having cutouts 6002 wherein tissue 74 was die or template cut and removed. The leaflets 68 cut from the tissue 74 of FIG. 17 are shown in FIG. 17A arranged in a petri dish 1200.

The templates or dies may be oversized to cut leaflets or valve portions that are larger than the size ultimately desired in the valve. Such oversizing may compensate for residual shrinkage of the tissue. In some embodiments, the leaflets or valve portions are 150% of the ultimate size desired in the valve. Or, in other words, the leaflets or valve portions are dimensionally larger by approximately 50 percent.

Valve Fabrication Conditions

Advantageously, the surface tension between the tissue, or any templated leaflets used the final assembly of the valve, and a smooth inert surface helps to control shrinkage during the valve assembly. In the present disclosure, exemplary three-leaflet living human engineered heart valve 50 can be fabricated keeping constructs wet with appropriate cell culture media containing HEPES and glucose.

Valve Fabrication

The present invention does not limit its scope by using one particular technique sequence in the preparation of the cardiac valve. It is implicit that different sequences could be used to form a wide variety of valvular sizes and shapes to satisfy functionality of a tissue-engineered living human valve. Further, each portion of the valve may be fabricated from a tissue-engineered tissue generated by a different technique or having different characteristics. For example, one leaflet may be cut from a planar tissue formed using a single step folding technique and another leaflet may be cut from a tubular tissue produced using a rolling technique. The leaflets can then be combined in the assembly of a single valve.

Overall, an exemplary tissue-engineered living human valve includes a plurality of tissue leaflets which are cut from a mature human tissue-engineered tissue. The leaflets are attached together to form a dimensionally stable and consistent coapting leaflet subassembly when subjected to physiological pressures. Then each of the leaflets of the subassembly is aligned with and individually sewn to a wireform, typically from the tip of one wireform commissure, uniformly around the leaflet cusp perimeter, to the tip of an adjacent wireform commissure. The wireform is usually covered with human tissue-engineered tissue but can alternatively be covered with cloth. The sewed sutures act like similarly aligned staples, all of which equally take toe loading force acting along the entire cusp of each of the pre-aligned leaflets. The resulting tissue-wireform structural assembly thereby formed reduces stress and potential fatigue at the leaflet suture interface by distributing stress evenly over the entire leaflet cusp from commissure to commissure.

Optionally, this improved, dimensionally stable, reduced stress assembly can be operatively attached to the top of a previously prepared stent. The stent is also typically covered with human tissue-engineered tissue but can alternatively be covered with cloth. It is desired to clamp the tissue leaflet cusps on a load-distributing tissue or cloth seat formed by the top of the covered stent without distorting the leaflets or disturbing their relative alignment and the resulting coaptation of their mating edges.

FIG. 18 is a top view of an embodiment of a living tissue-engineered human cardiac valve 50 of the present invention. Likewise, FIG. 19 is a perspective view of the embodiment illustrated in FIG. 18. FIG. 20 is an exploded assembly view, illustrating a few exemplary embodiments of a living tissue-engineered human cardiac valve 50 of the present invention. Illustrated are individual components of the valve 50 and alternative configurations produced in accordance with the teachings of the present invention. In the present disclosure, exemplary valve 50 is illustrated as a three-leaflet or tricuspid valve. However, it will be appreciated by those skilled in the art that valve 50 can be configured to have two leaflets or any other desired leaflet configuration depending on the intended application.

Valve 50 includes a pre-aligned, standardized leaflet subassembly 52, made from tissue-engineered tissue as described above, a tissue or cloth-covered wireform 54 and a support stent 56. As will be discussed in detail below, during assembly of valve 50, the pre-aligned leaflet subassembly 52 and the wireform 54 are first assembled in accordance with the present invention to form a tissue-wireform structural assembly 58. Then, the structural assembly 58 is optionally secured to a stent 56 to form the assembled valve 50.

As illustrated FIG. 20, valve 50 is uniquely configured to enable production of several useful alternative valves for a variety of end-use applications. For example, if the desired application is the replacement of a native heart valve, valve 50 can be attached to a relatively soft suture ring 60 for subsequent sewing into place within a heart (not shown). Alternatively, when desired, valve 50 can be attached to either an inflow conduit 64 and/or an outflow conduit 66.

Attachment of Leaflets to Each Other

A first step in the assembly of tissue valve 50 is the attachment of tissue leaflets 68 to one another to form a consistently dimensioned, standardized leaflet subassembly. Pre-alignment and stitching in accordance with the teachings of the present invention not only simplifies the manufacture of valve 50 but also functions to align the entire valve mating or seating surfaces at once. This eliminates variations in leaflet alignment and dimensional relationships and significantly minimizes the need to adjust the tissue leaflets after final assembly of the valve in order to ensure proper coaptation at the mating edges of the leaflets.

If one surface is smoother than the opposite surface, it is desirable that the less smooth surface be identified to serve as the mating surface at edge 70 with an adjacent leaflet edge 70. After the leaflets 68 are trimmed and the mating surfaces identified, two of the leaflets 68a, 68b are pre-aligned or mated together along with a template 69 as shown in FIG. 21. The two leaflets 68a, 68b are then attached or stitched together at one end 71 to define the first in a plurality of pairs of aligned, mating leaflet ends. For example, a needle that has been “double-threaded,” that is, needle 78 that has been threaded with a looped (or “folded”) segment of thread 80 is inserted and pushed through the leaflets 68a, 68b at the location dictated by guide slot 82 at one end of template 69. Template 69 can then be removed, with needle 78 being brought over the top of leaflets 68a, 68b and passed back through the loop and pulled tightly. Naturally, alternative attachment methods or stitches can be utilized. The opposite ends 72 of the first two leaflets 68a, 68b of the exemplary three leaflet valve are not sewn together at this time.

Referring now to FIG. 22, a third leaflet 68c is pre-aligned and attached to the other two leaflets 68a, 68b in a tricuspid format, again using template 69. In particular, third leaflet 68c is mated with template 69, and the respective unsewn ends 72 of the first two leaflets 68a, 68b are spread out and then aligned with the respective opposite ends 71, 72 of templated third leaflet 68c. Again using guide slot 82 of the template 69 as a guide, a double-threaded needle with thread 80 is inserted through each of the unsewn pairs of the three leaflets 68a, 68b, 68c to secure the leaflet ends together in pairs as shown. The template can then be removed, and, for each stitch, needle 78 can be brought over the top of leaflets 68a, 68b, 68c and passed back through the loop and pulled tightly to produce leaflet subassembly 52 having three leaflet mating ends.

During the making of the human tissue-engineered heart valve, appropriate forces sustaining the geometry of the construct such as additional stitches at the free edge of the construct, or surface tension can be used to avoid the shrinkage phenomena. For example, the shrinking of a construct can be controlled using surface tension by putting the construct in contact with pliable or rigid sewing aid. Alternatively, free edges of cusps can be sewn to prevent shrinking.

Attachment of Leaflets to Wireform

Referring now to FIGS. 23-24, it is preferred to attach leaflet subassembly 52 to the underside or bottom 83 of wireform 54. Exemplary wireform 54 is a wire covered with tissue-engineered tissue having a tissue edge 84 and is shaped in a manner substantially conforming to the shape of the leaflet subassembly structure 52. It may be appreciated that the wireform 54 may alternatively be covered with suitable cloth. In the embodiment shown, wireform 54 is generally circular in shape and has a sinusoidal undulation defining a plurality of commissure tips 86 corresponding to the pairs of leaflet mating ends. The covering of wireform 54 includes the circumferential tissue edge 84 which serves as a sewing or attachment surface for the leaflet subassembly 52. Exemplary wireform 54 includes the three raised commissure tips 86 which receive the three respective pairs of attached mating ends of leaflets 68a, 68b, and 68c of the pre-aligned leaflet subassembly 52.

An exemplary technique for attaching the leaflet pairs at an end of the leaflet subassembly 52 to one of the commissure tips 86 of wireform 54 is shown in FIG. 23. Needle 78 (not shown) with looped thread 80, which was used to sew the leaflet ends together, is inserted up from leaflets 68 (as shown in dashed lines), through an inner edge of tissue edge 84 as indicated at 87, so that the top surfaces of mating leaflets 68 are secured into contact with wireform 54. The needle is then re-inserted through an outer edge of and from underneath tissue edge 84 as indicated at 88′, and a first lock 89, preferably a single lock stitch, is made with thread 80. The locking process can be repeated as indicated at 88″ with a second lock 90, preferably a double lock stitch. Finally, the needle can be inserted into the middle of and from underneath tissue edge 84 as indicated at 91 and the thread pulled so that first and second locks 89, 90 are pulled underneath tissue edge 84 and thereby hidden and protected during the remaining fabrication process. The excess thread is then trimmed and discarded. This method is repeated for securing each of the respective pairs of attached, aligned mating leaflet ends of mated leaflets 68a, 68b, 68c of subassembly 52 to the respective commissure tips 86 of wireform 54. Thus, wireform 54 functions as an additional, permanent template for positioning the leaflet commissures in their final position relative to one another. As an added benefit of the present invention, this manufacturing technique further stabilizes the position of the coapting valve leaflets relative to one another prior to attachment of the leaflet cusps to the wireform. Thus, it is possible to attach the entire peripheral leaflet cusp uniformly from the tip of one commissure to the next in order to produce consistent attachment stress along the leaflet edge.

Referring now to FIGS. 24-25, the next exemplary step for securing the exemplary leaflet subassembly 52 to wireform 54 is to attach peripheral cusps 92 of each of the leaflets 68 to tissue edge 84. In that connection, slip knots 94 (i.e., knots which can be undone) are spaced periodically along wireform 54 to temporarily fit leaflet cusps 92 in position on wireform 54. Three of the slip knots 94 can be made for each leaflet cusp 92, with one at the center of the cusp and two at points of inflection with the commissures, as this helps to uniformly stabilize the cusp in position during attachment to wireform 54.

As shown in FIGS. 25-26, temporarily secured leaflet cusps 92 then are attached to wireform tissue edge 84, preferably using double-threaded “in-and-out” sutures 96, starting from a center position 98 of each leaflet cusp 92 and running to the tips of each commissure 86. At about one millimeter from the commissure tips 86, the threads are locked, buried and trimmed, preferably as described previously. Thus, unlike some tissue valves wherein leaflets are attached individually and the peripheral stitching of the cusps terminates before the tips of the commissures, producing a potential stress point, the produced valve assembly has uniform stitching from commissure tip to commissure tip and consistently aligned coapting leaflet mating edges.

Optional Attachment of Leaflet-Wireform Assembly to Support Stent

Once the assembled tissue-wireform structural assembly, which is identified by reference numeral 58, is produced as discussed above, the assembly 58 is then optionally attached to a support or stent 56. Referring to FIGS. 27-29, the tissue-wireform structural assembly 58 is first fitted onto the correspondingly configured stent 56 in a manner that will uniformly clamp the peripheral cusp edges of the leaflets 68 between an upper surface 99 (see FIG. 20) of stent 56 and the lower surface of wireform 54. This assembly technique further distributes stresses and loads of the leaflets 68 and contributes to their functional longevity. Moreover, pre-alignment of the leaflets 68 and attachment to the wireform 54 enables the dimensions of the entire valve 50 to be aligned at once and eliminates the dimensional variation that could occur with other techniques due to the utilization of separate commissure posts. In particular, stent 56 is dimensioned to mate or seat with the configuration of assembly 58, and assembly 58 is mated to stent 56 such that the lower surface of each commissure tip 86 of wireform 54 mates with the top surface of a corresponding and complementary stent commissure tip 100. Care is taken to ensure that central opening 102 formed by coapting mating leaflets 68 is not distorted while mating tissue-wireform structural assembly 58 to stent 56. Similarly, care is taken to ensure that leaflets 68 are uniformly clamped and remain evenly tensioned throughout this process.

Once wireform assembly 58 is mated to stent 56, a temporary pin 104 can be inserted at the bottom curve of each leaflet cusp 92 to temporarily secure wireform assembly 58 to stent 56. Stent 56 and assembly 58 then are sutured together as shown in FIGS. 28-29. Suturing of assembly 58 to stent 56 begins at the tops of the commissure tips 86. In particular, a double-threaded needle (not shown) is inserted through stent commissure tip 100 as indicated at 105′, between free tab ends 106, 108 of adjacent pairs of leaflets 68, and through tissue edge 84 of wireform assembly 58 as indicated at 109″. The needle is then inserted through the looped thread to form a single lock 110. A double lock 112 is then formed, with the needle being inserted through stent commissure tip at 105″ and through tissue edge 84 at 109″, substantially in the manner previously discussed so that double lock 112 is able to be pulled underneath tissue edge 84. Excess thread exiting from tissue edge 84 as indicated at 113 can then be trimmed and discarded. The identical procedure can be performed for the remaining commissure tips 86 of the wireform assembly 58. As a result, wireform commissure tips 86 evenly match with stent commissure tips 100.

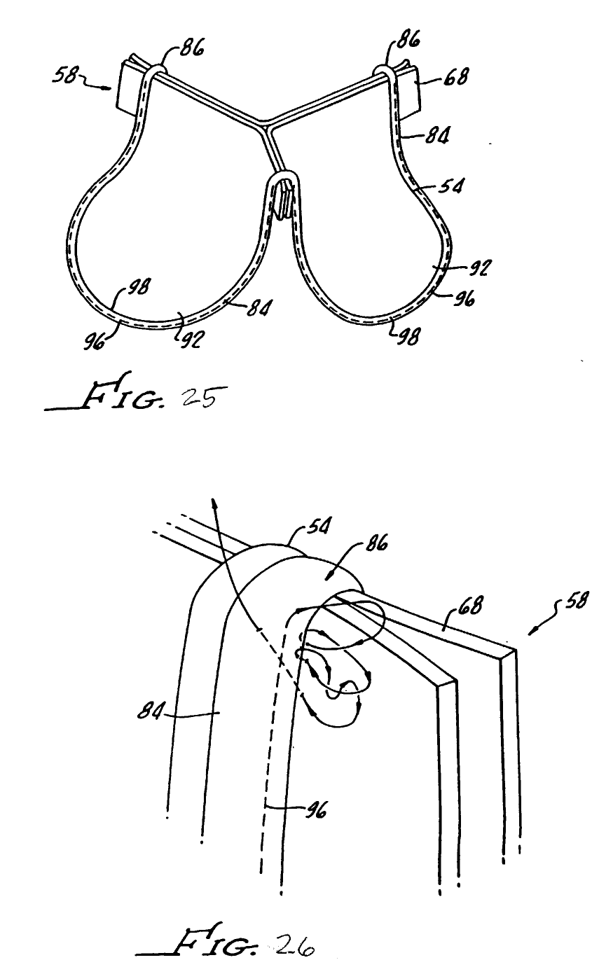

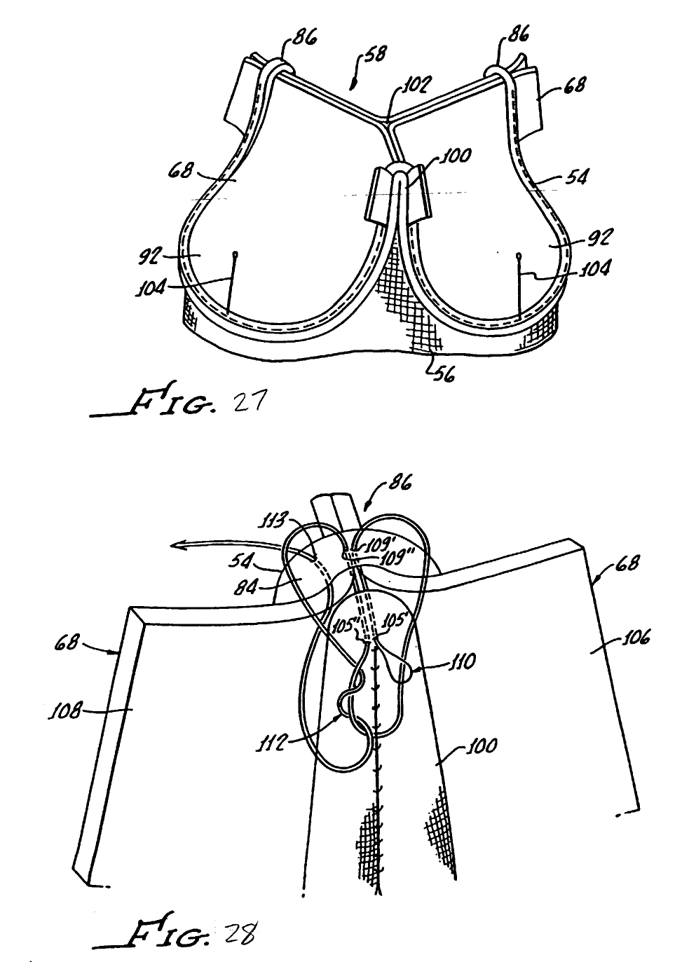

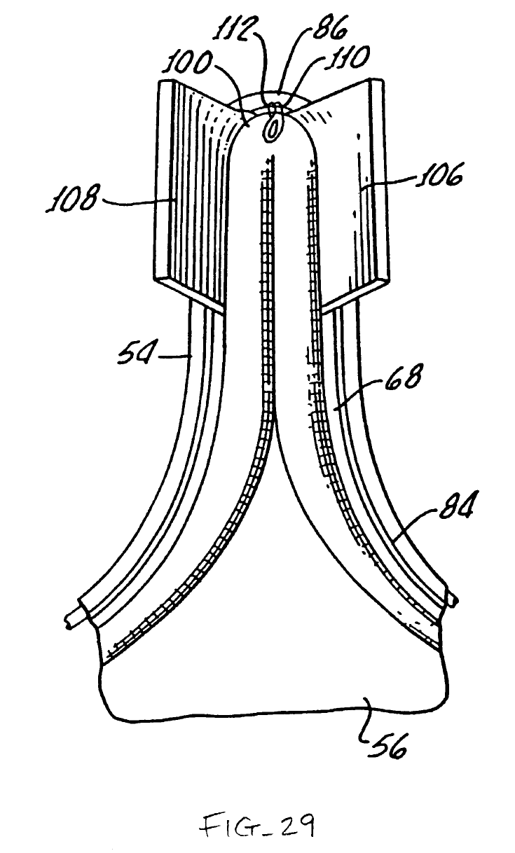

With reference to FIGS. 27, 30-32, the exemplary attachment procedure can be completed by inserting a double-threaded needle as previously described through stent 56 near the top of stent commissure tip 100 as indicated at 114′, through tissue leaflet 68 and through tissue edge 84 of wireform 54 as indicated at 115′. The needle is then re-inserted in a reverse manner through tissue edge at 115″, through stent commissure tip 100 at 114″ and passed through loop 115 of the double thread. With reference to FIG. 32, the suture is then tightened so that loop 115 is positioned securely and firmly against stent commissure tip 100. In-and-out suturing 116 (see also FIGS. 33-34) is then performed along the mating edges of stent 56 and wireform assembly 58 up to the next wireform assembly and stent commissure tips 86, 100. With reference to FIG. 31, at a position near the top of the commissure tip 86, a single lock 118 and a double lock 120 can be formed, and the thread can be buried beneath tissue edge 84 of wireform assembly 58 as described previously. It will be appreciated that the suturing just described can be initiated at any of the stent commissure tips 100 and that the in-and-out suturing 116 can be performed in either a clockwise or a counter-clockwise manner around the periphery of stent 56.

Upon completion of the in-and-out suturing 116 around the periphery of stent 56, the free tab ends 106, 108 of each pair of tissue leaflets 68 need to be secured to the respective stent commissure tip 100. Referring to FIGS. 33-34, two exemplary alternatives are provided to perform this task.

Referring to FIG. 33, a first exemplary alternative is to configure tab ends 106, 108 to form a butt joint 122. In particular, tab ends 106, 108 are trimmed such that, when folded towards each other, the respective end edges of each tab end 106, 108 mate evenly to form, preferably, a straight center line descending vertically from the top of commissure tip 100. The two leaflet tab ends 106, 108 are then stitched together with stitching 124.

Referring to FIG. 34, a second exemplary alternative for securing leaflet tab ends 106, 108 is to configure tab ends 106, 108 to mate evenly to form a flush junction 126 with tissue edge 84 of wireform 54 on either side of commissure tip 100. In particular, leaflet tab ends 106, 108 can be trimmed so that the end edges of each tab 106, 108 are sized to fit flush with tissue edge 84 of the wireform. Leaflet tab ends 106, 108 are then stitched to tissue edge 84 of wireform 54 with stitching 128 as shown. The alternative flush junction 126 so formed provides a somewhat flatter commissure than butt junction 122 of the first alternative, and, therefore, flush junction 126 can be more desirable when a more compact valve is needed. Both exemplary methods, however, allow even and reliable distribution of the load on the tissue leaflets at the commissures.

Construction of Stent

From the foregoing description, it will be appreciated that stent 56 is configured to have a structure suitable for mating and supporting wireform assembly 58. In that connection, an exemplary structure of stent 56 will now be described with reference to FIG. 35. Those skilled in the art will appreciate that the exemplary stent described herein is a multi-piece construction. However, it is contemplated as being within the scope of the present invention to provide a single-piece stent. However, the multi-piece stent assembly illustrated can make it easier to engineer or fine tune the radial stability of the stent while maintaining desirable axial flexibility of the commissure posts. The first step in the assembly of exemplary stent 56 is to fabricate an inner support member 130 and an outer support member 132, which, when mated together, generally form the shape of stent 56 which ultimately conforms to the configuration of wireform assembly 58. In the exemplary embodiments inner support member 130 is configured with three upstanding posts 134 which serve as the support structures for the stent commissure tips 100. Outer support member 132 also can include posts 136 that correspond to the posts 134 of the inner support member 130. However, posts 136 are truncated and therefore do not match the height of posts 134 on inner member 130. The inner and outer support members 130, 132 can be fabricated from a metal or plastic material depending on the desired characteristics of valve 50.

Disposed on inner support member 130 are a plurality of sewing holes 138 along the periphery of member 130 and on the posts 134. The outer support member 132 includes at least one sewing hole 139 on each of its truncated posts 136 which correspond with respective ones of the sewing holes 138 on each post 134 of the inner member 130. The inner diameter of outer support member 132 is sized to form a slip fit with the outer diameter of inner support member 130.

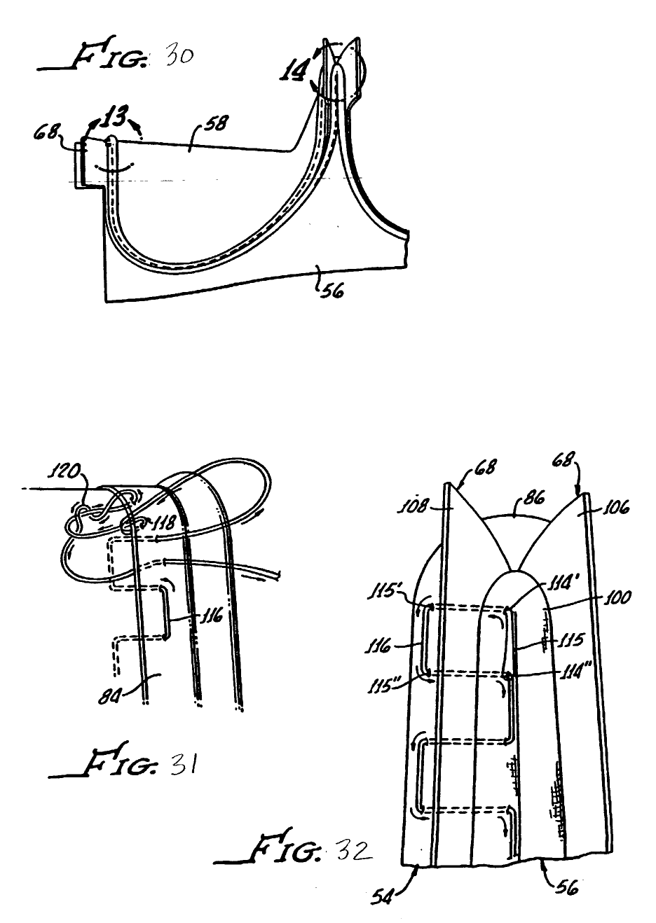

Inner support member 130 is placed within outer support member 132 such that sewing holes 139 of outer support member 132 align with sewing holes 138 on the respective posts 134 of inner member 130. The two members are then sewn together by inserting a double-threaded needle as described previously through the aligned holes 138, 139. As shown in FIG. 36, thread 140 inserted through each of the aligned holes 138, 439 is then passed through end loop 142 and tightened. The thread can then be locked using, for example, a slip knot (not shown), which is a knot that can slide along the thread to abut the support members. Accordingly, posts 134 of inner support member 130 flex to a greater extent from base portions thereof to tops thereof, and outer support member 132 augments the radial stability of inner support member 130, with the truncated posts 136 providing rigidity to base portions of posts 134 of inner support member 130.

Referring now to FIG. 37, once the inner and outer support members 130, 132 are sewn together, a covering material 144, preferably made from human engineered tissue, tissue or woven polyester, is cut and formed into a cylindrical tube for covering the combined support members 130, 132. Those skilled in the art will appreciate that the covering material is equally applicable to single-piece stent assemblies. Covering material 144 includes two crease lines 146, 148, the first of which, 146, is formed from folding an edge of material 144 to form a fold which receives posts 134 of inner support member 130. There is approximately 1 mm to 1.5 mm between first crease line 146 and a top edge 149 (see FIGS. 35-36) of each post 134 in the exemplary embodiment. Second crease line 148 is located such that it corresponds to a lower edge 150 (see FIG. 36) of combined support members 130, 132.

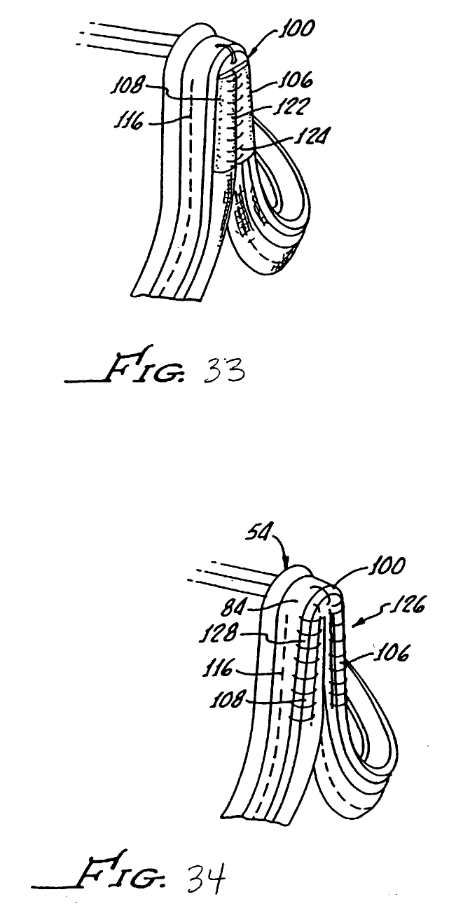

Referring now to FIG. 38, to secure covering material 144 to support members 130, 132, a threaded needle can be inserted through material 144, through a hole 151 of one of inner member posts 134, through the second layer of material 144 and then back through material 144 through the same hole 151 and through material 144. The needle then can be passed through a loop to form a first lock 152. This threading step can be performed up to two more times. The excess thread is then trimmed and discarded. The same procedure can be followed for each of the three posts 134 on inner support member 130.

Then, as shown in FIG. 39, the next exemplary step involves stitching covering material 144 to inner and outer support members 130, 132 along an upper edge 137 of inner support member 130. It can be appreciated that the covering material 144 can comprise any suitable material including human engineered tissue, tissue or cloth, to name a few. First, lower edge 154 of material 144 can be folded into the interior of support members 130, 132 along crease line 148 such that second crease line 148 defines the lower end or bottom of the support member structure. This fold results in dual-layered material 144 (including outer and inner material layers 156, 158) enveloping support members 130, 132. Then, using a single threaded needle, the layered material is stitched together at 155 along the curvature of the upper edge 153 of support members 130, 132. The stitching 155 is preferably backstitching, which is accomplished by inserting the needle a stitch length, for example, to the right and bringing it up an equal distance to the left. However, the stitching 155 does not extend to the tops 149 of posts 134, leaving a space of approximately 1 mm between the top 149 of post 134 and the stitching 155. After stitching the upper edge 153 of support members 130, 132, the material 144 then can be stitched in a similar manner at 156 along the lower edge 150 of support members 130, 132. The last stitch is then locked by tying a slip knot, which can be performed up to three times to lock the stitching securely in place.

Referring now to FIGS. 39-44, material 144 as now attached to support members 130, 132 is trimmed to conform to the shape of support members 130, 132 and, if desired, to provide a gasket-like sewing edge. To accomplish this, outer material layer 157 can be sliced downwardly from a top edge thereof to a distance approximately 5 mm to 6 mm above the top edge 153 of inner support member 130. In a similar manner, inner material layer 158 can be sliced downwardly from a top edge thereof to a distance approximately 2 mm to 3 mm above the bottom of the slice in outer material layer 157. The slices are made at a location midway between adjacent posts 134 of inner member 130 and are intended to align with one another in the downward direction, as indicated at 160.

Next, outer material layer 157 can be trimmed along the upper edge 153 of inner support member 130, starting at the bottom of the slice formed in outer material layer 157. In this exemplary embodiment of the present invention the trimming is performed in a manner such that the contour of the material 144 extends a distance of approximately 4 mm to 5 mm above the lower curved portions of the upper edge 153 of support member 130, a distance of approximately 2 mm to 3 mm above portions of support member 130 in the areas at or near the base of posts 134 of support member 130 and a distance of about 0.5 mm to 2 mm above the tops 149 of posts 134 of support member 130.

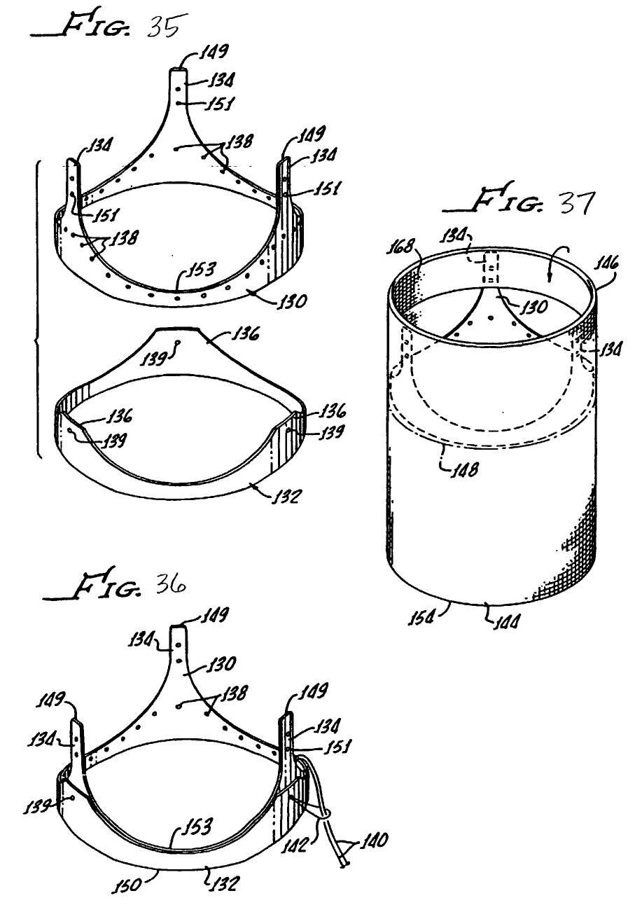

As shown in FIG. 40, inner material layer 158 is then folded over the tops 149 of posts 134 of inner member 130 and is anchored to posts 134 with a threaded needle stitched through sewing hole 151 in posts 134 in the manner previously described with respect to the upper folded section of material 144. However, after these locking stitches are executed, the needle is passed under the material so as to exit from the top of post 134.

Next, a series of trimming operations can be performed. Referring to FIGS. 40-41, a folded portion 162 of inner material layer 158 is trimmed around the entire circumference of the material so that lower edge 164 of folded portion 162 is approximately 1 mm to 1.5 mm from the stitch in hole 151 of post 134. A folded portion 168 of outer material layer 157 is folded over the tops 149 of post 134 of inner support member 130. Folded portion 162 of the inner material layer 158 is further trimmed so that its remaining edges, are flush with the edges of the previously trimmed inner material layer 158. With regard to the non-folded portion of inner material layer 158, this layer is trimmed in a manner such that its edges extend approximately 2 mm beyond the edges of the previously trimmed outer material layer 157. The 2 mm extension of the inner material layer 158 beyond the outer material layer 157 provides the material desired to form a seating and attachment or sewing surface on the stent.

Each of the trimming operations is performed starting from the central area between posts 134 of inner support member 130 to the tops 149 of posts 134. The arrangements of inner material layer 158, outer material fold 168, outer material layer 157 and inner material fold 162 are shown in the enlarged cross-section of FIG. 41.

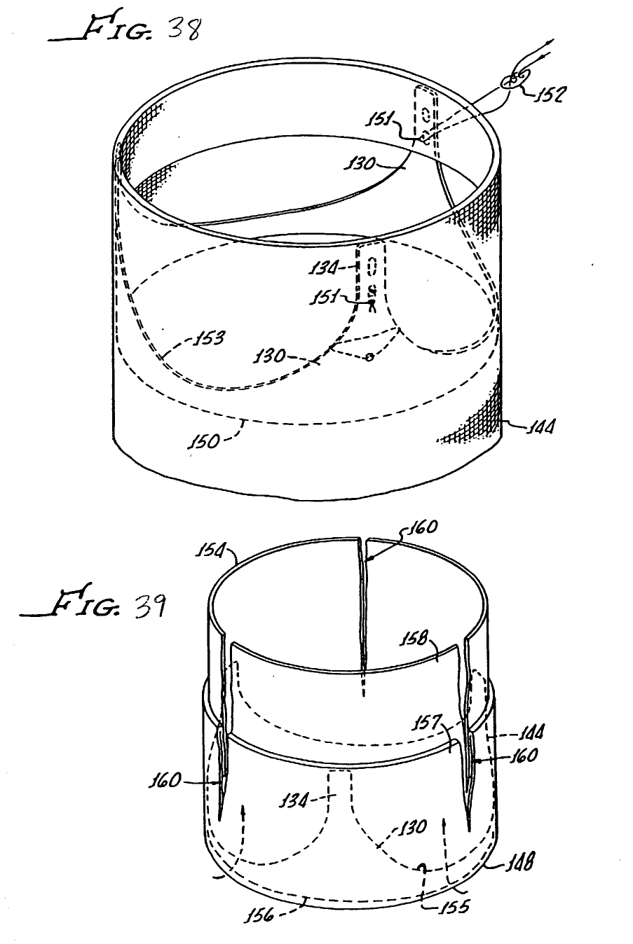

The remaining exemplary step to complete the assembly of the stent 56 is to fold and suture the material layers to form a sewing edge 169 around the stent 56. Referring to FIG. 42, inner material layer 158 is folded around post 134 and stitched so as to enclose post 134. More specifically, the thread previously inserted through the top of post 134 when connecting folded outer material layer 157 through sewing hole 151 is now used to create first and second locks 172 on the top of post 134 so as to hold inner material layer 158 in place on the top of post 134. A whipstitch 174 can then be utilized to further secure exemplary inner material layer 158 downwardly around post 134 approximately 8 mm from the top of post 134. When the bottom of the post 134 is reached, first and second locks are formed, and the thread is trimmed and discarded.

The above-described stitching operation is performed for each of the three posts 134. However, for the last of the posts 134 to be stitched, instead of trimming the thread after forming the first and second locks 172, untrimmed thread 176 can be used for performing the stitching of the material along the remaining edges of support members 130, 132 between posts 134.

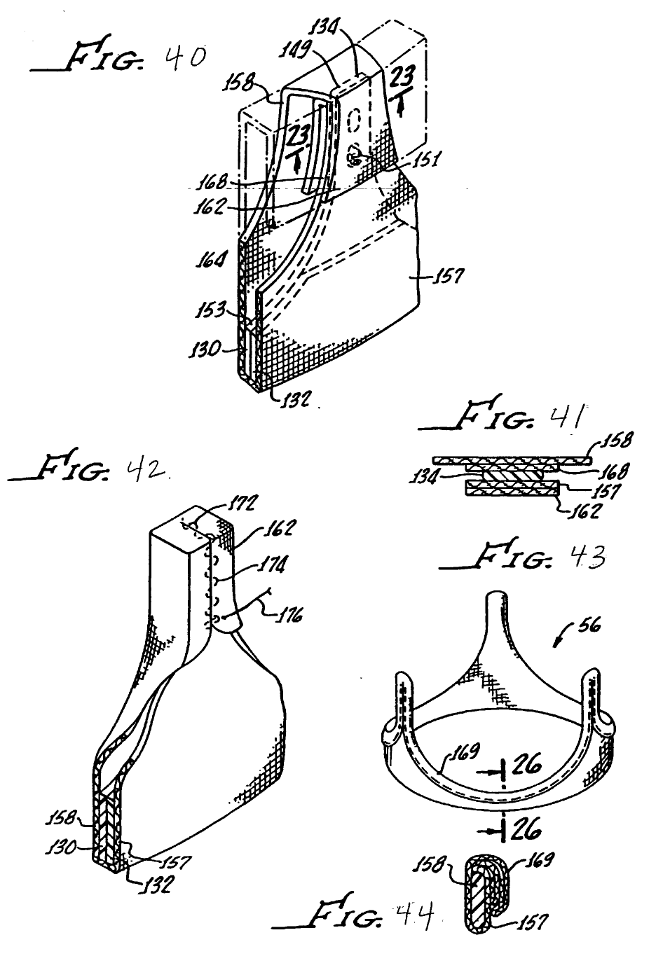

In that connection, with reference to FIGS. 43-44, inner material layer 158 is folded over the outer material layer 157, and an alternating stitching is applied to hold the folded layers in place on the support members and thereby to form the sewing edge 169 on the stent. After completing the stitching around the remaining portions of the support members 130, 132, a first and second lock stitch can be formed with the thread, and the excess thread is trimmed and discarded to complete the assembled stent 56.

Construction of Suture Ring

Where valve 50 is intended for use in the replacement of a native heart valve, a soft suture ring 60 can be used in completing the valve structure. For example, referring to FIG. 45, an exemplary ring washer or “remey” 180 is provided which is preferably made from non-woven polyester. Also provided is a silicone sponge waffle annulus 182 for mating with remey 180. In that connection, annulus 182 is configured to have a walled lip 184 configured to be disposed along the inner circumference 185 of remey 180. Lip 184 is contoured to include three depressions 186 that correspond with the lower curved surfaces between each commissure on valve 50. Remey 180 mounts on waffle annulus 182 such that remey 180 surrounds the walled lip 184. This produces a soft, relatively flexible, yet stable suture ring internal structure which, when covered with material as discussed below, functions as a compliant, stitchable interface between the natural tissues of the heart and the prosthetic tissue valve 50.

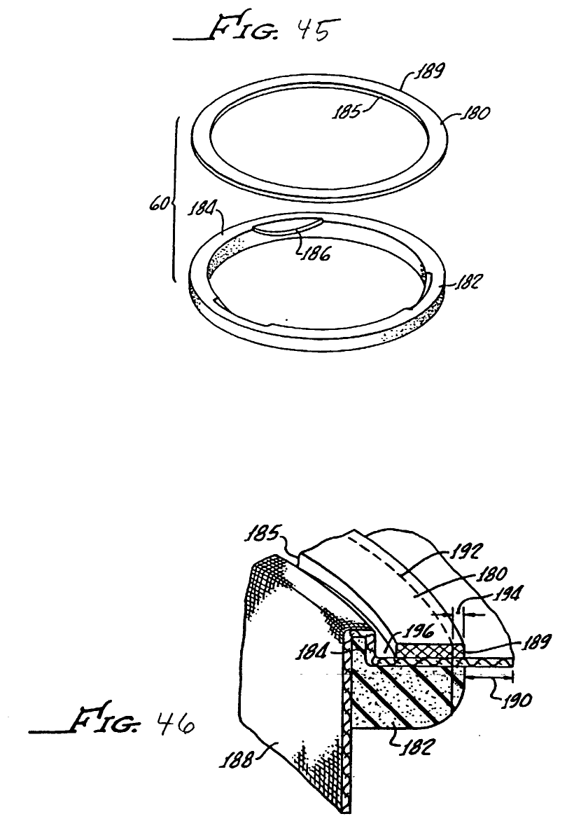

As shown in FIG. 46, before mounting remey 180 on waffle annulus 182, a material 188 is positioned around remey 180 to extend from the inner circumference 185 to the outer circumference 189. Remey 180 is then mounted on waffle annulus 182 such that material 188 is sandwiched between waffle annulus 182 and remey 180. Material 188 is placed to extend a distance 190 of approximately 3 mm to 5 mm beyond the outer circumferential edge 189 of remey 180, as shown in FIG. 46. Remey 180, material 188 and waffle annulus 182 are then sewn together using, for example, in-and-out suturing 192 around the circumference of remey 180. The exemplary suturing is preferably placed a distance 194 of approximately 1 mm from the outer circumferential edge 189 of remey 180. If desired, a second suture line (not shown) can be added at the same location as the first suture line, with each stitch of the second suture line placed between the stitches of the first suture line. The resulting suture 192 then appears as a continuous line of stitching. Additionally, as shown in FIG. 47, to further secure material 188 and waffle annulus 182 together, back stitching 195 can be applied in the space between the walled lip 184 of annulus 182 and remey 180, which space is indicated at 196 in FIG. 46.

Referring now to FIG. 48, material 188 can be attached to depressions 186 of the structural assembly of remey 180 and waffle annulus 182 with, for example, a single-threaded needle inserted at one corner 198 of depression 186 (through material 188 and annulus 182) and then with a double slip knot to secure the thread at corner 198. In-and-out stitching 200 can be then used to secure material 188 to the contour of depression 186. The same method can be followed for each depression 186. The excess material is then trimmed to the outer edge of remey 180 as indicated at 201.

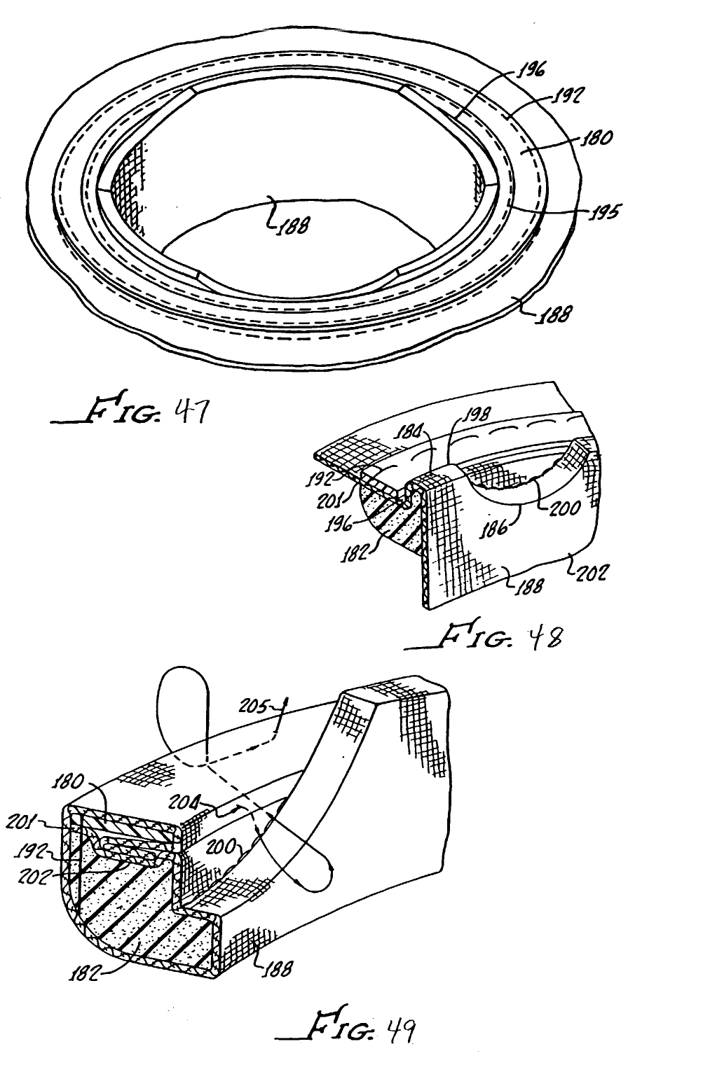

With additional reference to FIG. 49, an outer portion 202 of material 188 then can be folded around the external surfaces of remey 180 and tucked under remey 180 between remey 180 and waffle annulus 182. Because of annulus 182 is pliant, annulus 182 deforms and accommodates the outer portion 202 of material 188. Using a single-threaded needle, an alternating stitch 204 can be used to secure folded material 188 underneath remey 180. After completing the stitching of the entire circumference of remey 180, a double knot can be formed to secure the stitching, yielding a finished suture ring.

Optional Attachment of Valve to Suture Ring

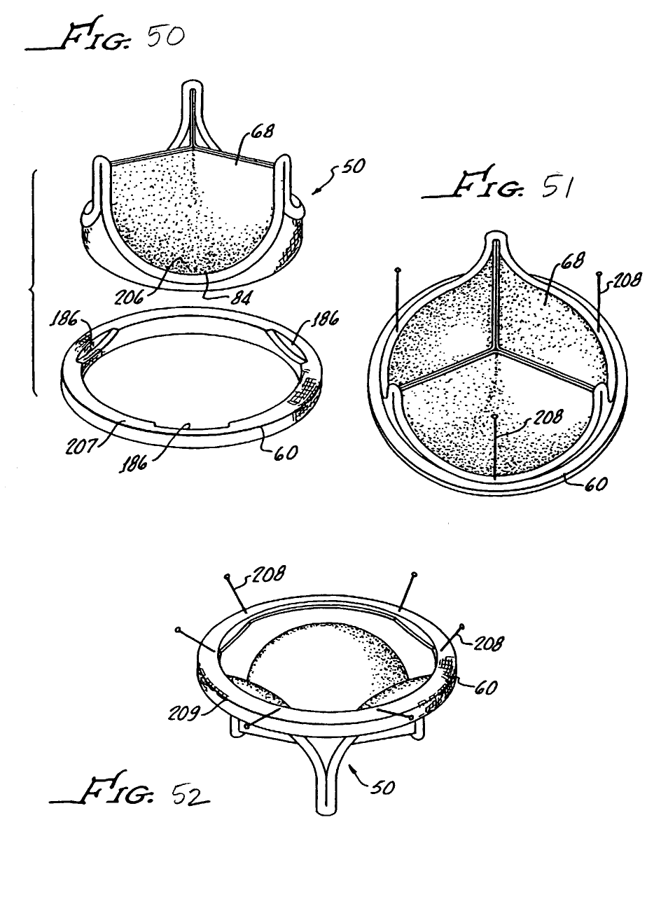

Referring to FIGS. 50-51, to attach suture ring 60 or an alternative structure such as flange 62 (see FIG. 20) to valve 50, depressions 186 of suture ring 60 are aligned with the descending peripheral cusps 206 of valve 50 and then mated together. More specifically, valve 50 is placed on suture ring 60 such that tissue edge 84 of the wireform 58 on the lower-most portion of each cusp on valve 50 is substantially flush with a top surface of suture ring 60 at corresponding depressions 186. Care is taken with the placement such that kinking or wrinkling of tissue leaflets 68 is avoided. Valve 50 can be temporarily pinned in place on suture ring 60 with needles 208 to facilitate this procedure.

As shown in FIG. 52, the assembly of pinned valve 50 and suture ring 60 can be flipped over, and suture ring 60 can be stitched to valve 50 along mating edges 209 of ring 60 and valve 50. More specifically, in the exemplary embodiment a single threaded needle can be used to sew suture ring 60 to the material of the stent structure. To facilitate the stitching step, the pieces are held temporarily, yet securely in place with additional needles 208. The opposite side of ring 60 and valve 50 can be sewn together in a similar manner.

Optional Attachment of Valve to Outflow Conduit

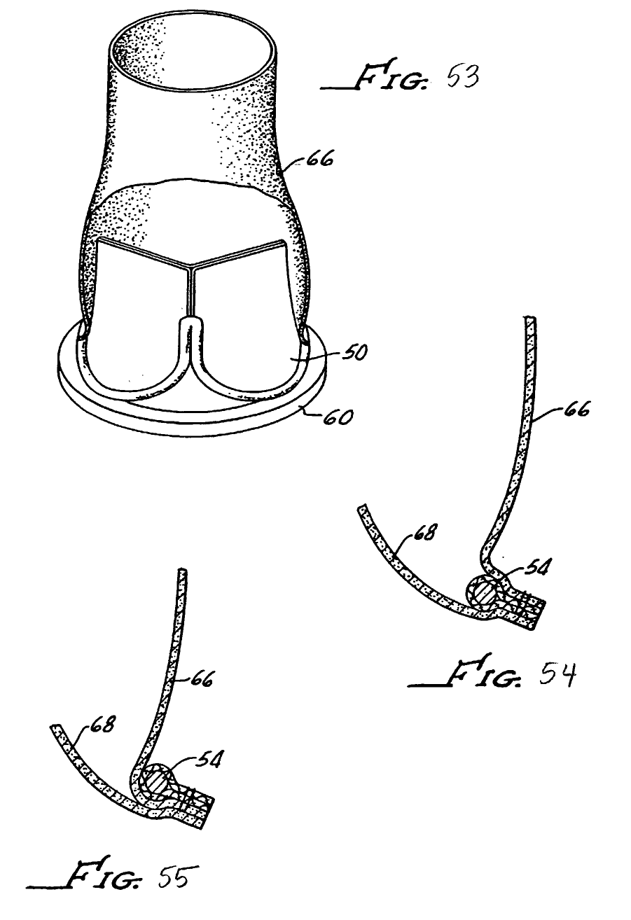

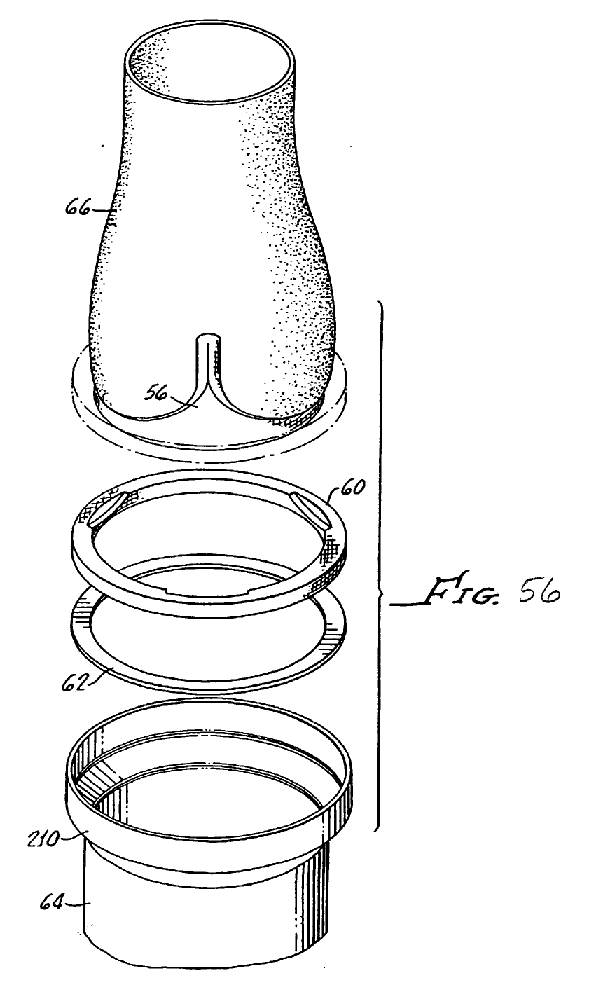

Referring now to FIGS. 53-55, in certain applications, it can be desirable to attach valve 50 to an outflow conduit such as that shown at 66. For example, in some patients requiring replacement of the aortic valve, a portion of the aorta itself can be damaged or diseased such that it needs replacement as well. Accordingly, consistent with the teachings of the present invention, the adaptable tissue valve structure can be modified to include an outflow conduit 66 which will function to replace the damaged aorta. Alternatively, in some intended mechanical pumping applications the adaptable tissue valve of the present invention can be provided with an outflow conduit to facilitate interfacing with the mechanical pumping structure. In either alternative, this can be accomplished as shown in FIGS. 53-54 where an outflow conduit 66 can be attached to wireform 54 at the time that the tissue leaflets 68 are being secured. In particular, referring to FIG. 54, conduit 66 can be secured on a side of wireform 54 opposite to tissue leaflets 68 by, for example, stitching. Alternatively, as shown in FIG. 55, conduit 66 can be stitched and secured to wireform 54 on the same side as tissue leaflets 68, or sandwiched therebetween. A third option is to simply secure conduit 66 to the periphery of the finished valve (not shown) as a subsequent sewing step. The valve 50 can be attached to an outflow conduit either with or without a sinus.

Optional Attachment of Valve to Inflow Side of Valve

FIG. 56 illustrates additional exemplary alternative options available for modification and attachment of valve 50. For example, as discussed above, when it is desired to use valve 50 as a conduit valve, suture ring 60 can be attached to valve 50 as previously described. Alternatively, in applications such as artificial hearts or left ventricular assist devices (LVADs), suture ring 60 is not necessarily required; hence, the lower end of stent 56 can be attached to flange 62 for use in mounting the valve in the artificial heart or LVAD.

Yet a further alternative adaptation involves those applications where an inflow conduit 64 is desired. In such applications, inflow conduit 64 can be attached directly to stent 56 of valve 50. More specifically, inflow conduit 64 can be configured to have a stepped circumference 210 which snugly mates with the outer periphery (or, alternatively, the inner periphery) of stent 56 and which can be sewn thereto. In this configuration, for example, in an artificial heart or an LVAD application, suture ring 60 could be attached to inflow conduit 64 rather than to valve 50.

Additional Embodiments

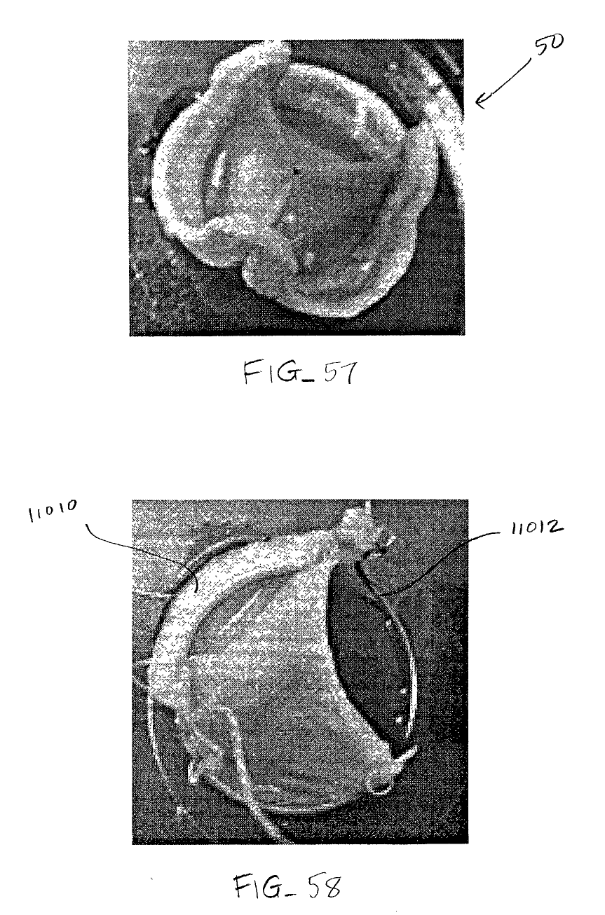

It may be appreciated that a variety of valves and related devices may be constructed with the use of the human engineered tissue described herein. For example, in addition to the valve described and illustrated above, a valve 50 shown in FIG. 57 may also be constructed. Here, the valve 50 has a more flexible valve design and is described in more detail in International Application Number PCT/US00/01855, incorporated by reference herein for all purposes. Likewise, the human engineered tissue of the present invention may also be used as a substitute for commonly used wireform cloth. Thus, as illustrated in FIG. 58, the human engineered tissue 11010 is shown covering wireform 11012 to create a natural living interface at the implantation site. Other similar uses are also within the scope of the present invention.

### EXAMPLE 1

### Preparation of a Reconstructed Multi-layered Human Tissue Construct from Sheets of Living Tissue Containing Fibroblasts and Extracellular Matrix Constituents

The following example describes one method for preparing a reconstructed multi-layered human tissue construct from sheets of living tissue containing fibroblasts and extracellular matrix constituents according to the present invention. All of the procedures described below are done under sterile conditions, preferably using a sterile flow hood. It can be appreciated that a variety of methods can be used to prepare the multi-layered tissue construct and this example is not intended to limit the scope of this invention to the number of sheets of tissue superimposed, to one particular shape (i.e., thickness and size), cell type, origin, age, maturation time, component concentration, and culture conditions to generate the multi-layered human tissue construct. One skilled in the art can readily appreciate that various modifications can be made to the method without departing from the scope and spirit of the invention.

Typically, 750,000 viable sub-cultured human skin fibroblasts are seeded in a standard 75 cm² sterile Petri dish for a final seeding density of 104 cells/cm². Cells are fed with culture medium (DME containing 10% fetal calf serum (FCS), 100 IU/ml penicillin and 25 ug/ml gentamicin), and cultivated for 4 weeks to form sheets that can be manipulated. The culture medium is changed three times per week. A freshly prepared solution of ascorbic acid is added each time the medium is changed to obtain a final concentration of 50 μg/ml of ascorbic acid. Cells are kept in a humidified atmosphere (92% air and 8% CO₂) throughout the culture. After the sheets of tissue are peeled from the dishes, three separate sheets of living tissue are superimposed. Stainless steel ingots (approximately 1 mm×2 mm×8 mm) are used and placed around the tissue sheet perimeter to keep the tissue construct anchored and stretched to its maximal area on the surface of the petri dish. Another sheet of tissue is then placed on top of the first tissue sheet. One by one, the ingots are carefully pushed aside from the first sheet and other ingots are placed around the tissue sheet perimeter of the second layer, spreading it over the first sheet of tissue. These steps are repeated to obtain a three-layered tissue construct.

A semi-rigid sponge permeable to the culture media is then cut to fit the size of the tissue construct between the ingots and applied to the surface of the construct. The sponge should closely fit the perimeter delimited by the ingots, but not overlap or exceed it. Ingots are then evenly distributed on the sponge surface to put some weight on it. The sponge as well as the ingots is removed 24 to 72 hrs following the stacking. Seven days after the stacking of the sheets of tissue, three three-layered tissue constructs were superimposed to form the final nine-layered tissue construct using the same technique as described above. The constructs were further incubated for 6 weeks and culture medium refreshed 3 times a week. The tissue constructs are then ready for shipment processing.

Although the foregoing invention has been described in detail for purposes of clarity of understanding, it will be obvious that certain modifications can be practiced within the scope of the appended claims. All publications and patent documents cited herein are hereby incorporated by reference in their entirety for all purposes to the same extent as if each were so individually denoted.

What is claimed is:

1. A human engineered tissue-type heart valve comprising:
a plurality of leaflets assembled to form a heart valve,
wherein each leaflet is comprised of at least five layers of at least one living tissue sheet fused together to form a self-supporting human engineered tissue.

2. A human engineered tissue-type heart valve as in claim 1, wherein the at least one living tissue sheet is formed from an extracellular matrix secreted by mesenchymal cells.

3. A human engineered tissue-type heart valve as in claim 2, wherein the mesenchymal cells are allogeneic, autologous, genetically-modified or a combination of these.

4. A human engineered tissue-type heart valve as in claim 2, wherein the mesenchymal cells comprise dermal fibroblasts and adventitial fibroblasts.

5. A human engineered tissue-type heart valve as in claim 2, wherein the mesenchymal cells comprise myofibroblasts.

6. A human engineered tissue-type heart valve as in claim 2, wherein the mesenchymal cells comprise interstitial valvular cells, endothelial cells or a combination of these.

7. A human engineered tissue-type heart valve as in claim 1, wherein the at least one living tissue sheet is formed from an extracellular matrix secreted by embryonic, post-natal or adult stem cells.

8. A human engineered tissue-type heart valve as in claim 7, wherein the stem cells are allogeneic, autologous, genetically-modified or a combination of these.

9. A human engineered tissue-type heart valve as in claim 1, wherein each leaflet is comprised of at least seven layers.

10. A human engineered tissue-type heart valve as in claim 9, wherein each leaflet is comprised of at least nine layers.

11. A human engineered tissue-type heart valve as in claim 1, wherein the at least five layers of at least one living tissue sheet comprises at least five living tissue sheets stacked on top of each other.

12. A human engineered tissue-type heart valve as in claim 1, wherein the at least five layers of at least one living tissue sheet comprises one living tissue sheets folded to create five layers.

13. A human engineered tissue-type heart valve as in claim 1, wherein the human engineered tissue has a thickness in the range of approximately 0.1 mm to 0.6 mm.

14. A human engineered tissue-type heart valve as in claim 13, wherein the human engineered tissue has a thickness in the range of approximately 0.3 mm to 0.6 mm.

15. A human engineered tissue-type heart valve as in claim 1, wherein the at least one living tissue sheet includes collagen type I, collagen type III, elastin, glycosaminoglycans, growth factors, glycoproteins and water.

16. A human engineered tissue-type heart valve comprising:
a plurality of leaflets assembled to form a heart valve,
wherein each leaflet is comprised of layers of at least one living tissue sheet fused together to form a self-supporting human engineered tissue having a thickness of at least approximately 0.16 mm.

17. A human engineered tissue-type heart valve comprising:
a plurality of leaflets arranged to form a heart valve,
wherein each leaflet is comprised of layers of at least one allogeneic living tissue sheet fused together to form a self-supporting human engineered tissue which undergoes living cell replacement upon implantation in a patient so that at least some of the allogeneic cells are replaced with the patient's living cells.

18. A human engineered tissue-type heart valve as in claim 17, wherein the majority of the allogenic cells are replaced with the patient's living cells.

19. A human engineered tissue-type heart valve as in claim 18, wherein approximately all of the allogenic cells are replaced with the patient's living cells.

20. A human engineered tissue-type heart valve as in claim 17, wherein the self-supporting human engineered tissue undergoes remodeling upon implantation in the patient.

21. A human engineered tissue-type heart valve as in claim 17, wherein the allogeneic cells comprise mesenchymal cells.

22. A human engineered tissue-type heart valve as in claim 21, wherein the mesenchymal cells comprise dermal fibroblasts or adventitial fibroblasts.

23. A human engineered tissue-type heart valve as in claim 21, wherein the mesenchymal cells comprise interstitial valvular cells, myofibroblasts, endothelial cells or a combination of any of these.

24. A human engineered tissue-type heart valve as in claim 17, wherein the allogeneic cells comprise embryonic, post-natal or adult stem cells.

25. A human engineered tissue-type heart valve as in claim 17, wherein each leaflet is comprised of at least five layers.

26. A human engineered tissue-type heart valve as in claim 17, wherein each leaflet has a thickness in the range of approximately 0.1 mm to 0.6 mm.

27. A human engineered tissue-type heart valve as in claim 26, wherein each leaflet has a thickness in the range of approximately 0.3 mm to 0.6 mm.

28. A method of making a human engineered heart valve, the method comprising:
generating at least one living tissue sheet by secreting an extracellular matrix from cells;
layering the at least one living tissue sheet to form a layered construct having at least seven layers; and
culturing the layered construct to fuse the layers to form a human engineered tissue.

29. A method as in claim 28, wherein layering the at least one living tissue sheet comprises stacking a plurality of individual sheets on top of each other.

30. A method as in claim 28, wherein layering the at least one living tissue sheet comprises folding a single sheet upon itself.

31. A method as in claim 28, wherein layering the at least one living tissue sheet comprises creating enough layers so that the human engineered tissue has a thickness in the range of approximately 0.1 mm to 0.6 mm.

32. A method as in claim 31, wherein layering the at least one living tissue sheet comprises creating enough layers so that the human engineered tissue has a thickness in the range of approximately 0.3 mm to 0.6 mm.

33. A method as in claim 28, wherein culturing comprises exposing the layered construct to L-ascorbate acid or a phosphate derivative of L-ascorbate acid serum.

34. A method as in claim 28, wherein culturing comprises anchoring the layered construct to reduce shrinkage.

35. A method as in claim 28, wherein forming the plurality of leaflets from the human engineered tissue comprises cutting each leaflet shape out of the human engineered tissue.

36. A method as in claim 28, wherein the cells comprise mesenchymal cells.

37. A method as in claim 28 wherein the cells comprise embryonic, post-natal or adult stem cells.

38. A method of preparing human engineered tissue for use in making a heart valve, the method comprising:
generating at least one living tissue sheet by secreting an extracellular matrix from cells;
layering the at least one living tissue sheet to form a layered construct;
culturing the layered construct to fuse the layers to form the human engineered tissue; and
regulating shrinkage of the human engineered tissue.

39. A method as in claim 38, wherein regulating shrinkage comprises anchoring the human engineered tissue.

40. A method as in claim 38, wherein anchoring comprises placing a plurality of anchors upon the human engineered tissue.

41. A method as in claim 40, wherein anchors are placed in a generally rectangular shape.

42. A method as in claim 40, wherein anchors are placed in a generally circular or oval shape.

43. The method of claim 38, wherein regulating shrinkage comprises maintaining the human engineered tissue in wet conditions.

44. The method of claim 40, wherein wet conditions comprises wet with HEPES, high glucose and Dulbecco Modified Eagle Medium.

45. The method of claim 38, wherein regulating shrinkage comprises creating a surface adhesion on the human engineered tissue to reduce shrinkage.

46. A method of preparing human engineered tissue for use in making a heart valve, the method comprising:
generating at least one living tissue sheet by secreting an extracellular matrix from cells;
layering the at least one living tissue sheet to form a layered construct;
culturing the layered construct to fuse the layers to form the human engineered tissue; and
cutting a leaflet shape out of the human engineered tissue which is dimensionally larger than a desired leaflet shape to account for shrinkage.

47. A method as in claim 46, wherein cutting each leaflet shape comprises punch cutting with a die having the leaflet shape.

48. A method as in claim 46, wherein cutting each leaflet shape comprises cutting around a template having the leaflet shape.

49. A method as in claims 46, wherein dimensionally larger is approximately 50 percent larger.

50. The method of claim 46, further comprising constructing a heart valve using the leaflet shape.

51. A human engineered tissue-type heart valve comprising:
a tissue leaflet subassembly mated with a wireform to form a heart valve,
wherein each leaflet is comprised of at least five layers of at least one living tissue sheet fused together to form a self-supporting human engineered tissue, and
wherein at least a portion of the wireform is covered with the tissue.

52. A human engineered tissue-type heart valve as in claim 51, wherein the heart valve further comprises a support stent mated with the wireform.

53. A human engineered tissue-type heart valve as in claim 52, wherein at least a portion of the support stent is covered with the tissue.

54. A human engineered tissue-type heart valve as in claim 52, wherein the heart valve further comprises an adaptable structural interface attached to the support stent.

55. A human engineered tissue-type heart valve as in claim 54, wherein at least a portion of the adaptable structural interface is covered with the tissue.
